SEQUENCE LISTING (33 sequences)

SEQ ID NO: 1 - DNA, 882 bp, Staphylococcus aureus
  source 1..882: mol_type = genomic DNA
  CDS 4..882
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaat

SEQ ID NO: 2 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 3 - DNA, 882 bp, synthetic construct
  misc_feature 1..882: note = DNA Sequence encoding one subunit of alpha-HL M113R/N139Q
  source 1..882: mol_type = other DNA
  CDS 4..882
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtataggagtactttaacttatggattcaacggtaatgttactggtgatgatacaggaaaaattggcggccttattggtgcacaagtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaat

SEQ ID NO: 4 - protein, 293 aa, synthetic construct
  REGION 1..293: note = Synthetic Construct
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYRSTLTYGFNGNVTGDDTGKIGGLIGAQVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - DNA, 4543 bp, synthetic construct
  misc_feature 1..4543: note = DNA Sequence encoding pT7-SC1_BspEI-KO
  source 1..4543: mol_type = other DNA
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctagcccgcctaatgagcgggcttttttttagatctcgatcccgcgaaattaatacgactcactatagggagaccacaacggtttccctctagaaataattttgtttaactttaagaaggagatatacatatggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattaatgtaaattatttgtacatgtacaaataaatataatttataactttagccgaaagcttggatccggctgctaacaaagcccgaaaggaagctgagttggctgctgccaccgctgagcaataactagcataaccccttggggcctctaaacgggtcttgaggggttttttgctgaaaggaggaactatatataattcgagctcggtacccaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccagtattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggatgccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggatcctctagagtcgacctgcaggcatgcaagctatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctagcttatcgatgataagctgtcaaacatgagaa

SEQ ID NO: 6 - DNA, 888 bp, synthetic construct
  misc_feature 1..888: note = DNA Sequence encoding one subunit of a wild-type alpha-hemolysin containing a BspEI cloning site at position 1 (L1)
  source 1..888: mol_type = other DNA
atggcagattctgatattaatattaaaaccggtactacagatattggaagcaatacttccggaacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaat

SEQ ID NO: 7 - DNA, 888 bp, synthetic construct
  misc_feature 1..888: note = DNA Sequence encoding one subunit of a wild-type alpha-hemolysin containing a BspEI cloning site at position 2 (L2a)
  source 1..888: mol_type = other DNA
atggcagattctgatattaatattaaaaccggtactacagatattggaagcaatactacagtaaaaacaggtgatttagtcacttatgataaagaaaatggcatgcacaaaaaagtattttatagttttatcgattccggagataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaat

SEQ ID NO: 8 - DNA, 888 bp, synthetic construct
  misc_feature 1..888: note = DNA Sequence encoding one subunit of a wild-type alpha-hemolysin containing a BspEI cloning site at position 2 (L2b)
  source 1..888: mol_type = other DNA
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaatccggaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaat

SEQ ID NO: 9 - DNA, 804 bp, Escherichia coli
  source 1..804: mol_type = genomic DNA
  CDS 1..804
atgaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggatatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgc

SEQ ID NO: 10 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 11 - DNA, 1425 bp, Escherichia coli
  source 1..1425: mol_type = genomic DNA
  CDS 1..1425
atgatgaatgacggtaagcaacaatctacctttttgtttcacgattacgaaacctttggcacgcaccccgcgttagatcgccctgcacagttcgcagccattcgcaccgatagcgaattcaatgtcatcggcgaacccgaagtcttttactgcaagcccgctgatgactatttaccccagccaggagccgtattaattaccggtattaccccgcaggaagcacgggcgaaaggagaaaacgaagccgcgtttgccgcccgtattcactcgctttttaccgtaccgaagacctgtattctgggctacaacaatgtgcgtttcgacgacgaagtcacacgcaacattttttatcgtaatttctacgatccttacgcctggagctggcagcatgataactcgcgctgggatttactggatgttatgcgtgcctgttatgccctgcgcccggaaggaataaactggcctgaaaatgatgacggtctaccgagctttcgccttgagcatttaaccaaagcgaatggtattgaacatagcaacgcccacgatgcgatggctgatgtgtacgccactattgcgatggcaaagctggtaaaaacgcgtcagccacgcctgtttgattatctctttacccatcgtaataaacacaaactgatggcgttgattgatgttccgcagatgaaacccctggtgcacgtttccggaatgtttggagcatggcgcggcaataccagctgggtggcaccgctggcgtggcatcctgaaaatcgcaatgccgtaattatggtggatttggcaggagacatttcgccattactggaactggatagcgacacattgcgcgagcgtttatataccgcaaaaaccgatcttggcgataacgccgccgttccggttaagctggtgcatatcaataaatgtccggtgctggcccaggcgaatacgctacgcccggaagatgccgaccgactgggaattaatcgtcagcattgcctcgataacctgaaaattctgcgtgaaaatccgcaagtgcgcgaaaaagtggtggcgatattcgcggaagccgaaccgtttacgccttcagataacgtggatgcacagctttataacggctttttcagtgacgcagatcgtgcagcaatgaaaattgtgctggaaaccgagccgcgtaatttaccggcactggatatcacttttgttgataaacggattgaaaagctgttgttcaattatcgggcacgcaacttcccggggacgctggattatgccgagcagcaacgctggctggagcaccgtcgccaggtcttcacgccagagtttttgcagggttatgctgatgaattgcagatgctggtacaacaatatgccgatgacaaagagaaagtggcgctgttaaaagcactttggcagtacgcggaagagattgtc

SEQ ID NO: 12 - protein, 475 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIV

SEQ ID NO: 13 - DNA, 1275 bp, Thermus thermophilus
  source 1..1275: mol_type = genomic DNA
  CDS 1..1275
atgtttcgtcgtaaagaagatctggatccgccgctggcactgctgccgctgaaaggcctgcgcgaagccgccgcactgctggaagaagcgctgcgtcaaggtaaacgcattcgtgttcacggcgactatgatgcggatggcctgaccggcaccgcgatcctggttcgtggtctggccgccctgggtgcggatgttcatccgtttatcccgcaccgcctggaagaaggctatggtgtcctgatggaacgcgtcccggaacatctggaagcctcggacctgtttctgaccgttgactgcggcattaccaaccatgcggaactgcgcgaactgctggaaaatggcgtggaagtcattgttaccgatcatcatacgccgggcaaaacgccgccgccgggtctggtcgtgcatccggcgctgacgccggatctgaaagaaaaaccgaccggcgcaggcgtggcgtttctgctgctgtgggcactgcatgaacgcctgggcctgccgccgccgctggaatacgcggacctggcagccgttggcaccattgccgacgttgccccgctgtggggttggaatcgtgcactggtgaaagaaggtctggcacgcatcccggcttcatcttgggtgggcctgcgtctgctggctgaagccgtgggctataccggcaaagcggtcgaagtcgctttccgcatcgcgccgcgcatcaatgcggcttcccgcctgggcgaagcggaaaaagccctgcgcctgctgctgacggatgatgcggcagaagctcaggcgctggtcggcgaactgcaccgtctgaacgcccgtcgtcagaccctggaagaagcgatgctgcgcaaactgctgccgcaggccgacccggaagcgaaagccatcgttctgctggacccggaaggccatccgggtgttatgggtattgtggcctctcgcatcctggaagcgaccctgcgcccggtctttctggtggcccagggcaaaggcaccgtgcgttcgctggctccgatttccgccgtcgaagcactgcgcagcgcggaagatctgctgctgcgttatggtggtcataaagaagcggcgggtttcgcaatggatgaagcgctgtttccggcgttcaaagcacgcgttgaagcgtatgccgcacgtttcccggatccggttcgtgaagtggcactgctggatctgctgccggaaccgggcctgctgccgcaggtgttccgtgaactggcactgctggaaccgtatggtgaaggtaacccggaaccgctgttcctg

SEQ ID NO: 14 - protein, 425 aa, Thermus thermophilus
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 15 - DNA, 738 bp, unidentified
  source 1..738: mol_type = genomic DNA; note = Bacteriophage lambda
  CDS 31..708
tccggaagcggctctggtagtggttctggcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatctggctctggttccggcagcggttccgga

SEQ ID NO: 16 - protein, 226 aa, unidentified
  source 1..226: note = Bacteriophage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 17 - DNA, 1794 bp, synthetic construct
  misc_feature 1..1794: note = DNA Sequence encoding HL-wt-EcoExoIII-L1-H6
  source 1..1794: mol_type = other DNA
  CDS 4..1794
atggcagattctgatattaatattaaaaccggtactacagatattggaagcaatacttccggaagcggctctggtagtggttctggcatgaaatttgttagcttcaatatcaacggcctgcgcgcgcgcccgcatcagctggaagcgattgtggaaaaacatcagccggatgttattggtctgcaggaaaccaaagttcacgatgatatgtttccgctggaagaagtggcgaaactgggctataacgtgttttatcatggccagaaaggtcattatggcgtggccctgctgaccaaagaaaccccgatcgcggttcgtcgtggttttccgggtgatgatgaagaagcgcagcgtcgtattattatggcggaaattccgagcctgctgggcaatgtgaccgttattaacggctattttccgcagggcgaaagccgtgatcatccgattaaatttccggccaaagcgcagttctatcagaacctgcagaactatctggaaaccgaactgaaacgtgataatccggtgctgatcatgggcgatatgaacattagcccgaccgatctggatattggcattggcgaagaaaaccgtaaacgctggctgcgtaccggtaaatgcagctttctgccggaagaacgtgaatggatggatcgcctgatgagctggggcctggtggatacctttcgtcatgcgaacccgcagaccgccgatcgctttagctggtttgattatcgcagcaaaggttttgatgataaccgtggcctgcgcattgatctgctgctggcgagccagccgctggcggaatgctgcgttgaaaccggtattgattatgaaattcgcagcatggaaaaaccgagcgatcacgccccggtgtgggcgacctttcgccgctctggctctggttccggcagcggttccggaacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaatggtggttcgggctcatctggtggctcgagtcaccatcatcatcaccac

SEQ ID NO: 18 - protein, 597 aa, synthetic construct
  REGION 1..597: note = Synthetic Construct
ADSDINIKTGTTDIGSNTSGSGSGSGSGMKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRRSGSGSGSGSGTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGGSGSSGGSSHHHHHH

SEQ ID NO: 19 - DNA, 1794 bp, synthetic construct
  misc_feature 1..1794: note = DNA Sequence encoding HL-RQC-EcoExoIII-L1-H6
  source 1..1794: mol_type = other DNA
  CDS 4..1794
atggcagattctgatattaatattaaaaccggtactacagatattggaagcaatacttccggaagcggctctggtagtggttctggcatgaaatttgttagcttcaatatcaacggcctgcgcgcgcgcccgcatcagctggaagcgattgtggaaaaacatcagccggatgttattggtctgcaggaaaccaaagttcacgatgatatgtttccgctggaagaagtggcgaaactgggctataacgtgttttatcatggccagaaaggtcattatggcgtggccctgctgaccaaagaaaccccgatcgcggttcgtcgtggttttccgggtgatgatgaagaagcgcagcgtcgtattattatggcggaaattccgagcctgctgggcaatgtgaccgttattaacggctattttccgcagggcgaaagccgtgatcatccgattaaatttccggccaaagcgcagttctatcagaacctgcagaactatctggaaaccgaactgaaacgtgataatccggtgctgatcatgggcgatatgaacattagcccgaccgatctggatattggcattggcgaagaaaaccgtaaacgctggctgcgtaccggtaaatgcagctttctgccggaagaacgtgaatggatggatcgcctgatgagctggggcctggtggatacctttcgtcatgcgaacccgcagaccgccgatcgctttagctggtttgattatcgcagcaaaggttttgatgataaccgtggcctgcgcattgatctgctgctggcgagccagccgctggcggaatgctgcgttgaaaccggtattgattatgaaattcgcagcatggaaaaaccgagcgatcacgccccggtgtgggcgacctttcgccgctctggctctggttccggcagcggttccggaacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtataggagtactttaacttatggattcaacggtaatgttactggtgatgatacaggaaaaattggcggctgtattggtgcacaagtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaatggtggttcgggctcatctggtggctcgagtcaccatcatcatcaccac

SEQ ID NO: 20 - protein, 597 aa, synthetic construct
  REGION 1..597: note = Synthetic Construct
ADSDINIKTGTTDIGSNTSGSGSGSGSGMKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRRSGSGSGSGSGTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYRSTLTYGFNGNVTGDDTGKIGGCIGAQVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGGSGSSGGSSHHHHHH

SEQ ID NO: 21 - DNA, 2415 bp, synthetic construct
  misc_feature 1..2415: note = DNA Sequence encoding HL-RQC-EcoExoI-L1-H6
  source 1..2415: mol_type = other DNA
  CDS 4..2415
atggcagattctgatattaatattaaaaccggtactacagatattggaagcaatacttccggaagcggctctggtagtggttctggcatgatgaacgatggcaaacagcagagcaccttcctgtttcatgattatgaaaccttcggtacccatccggccctggatcgtccggcgcagtttgcggccattcgcaccgatagcgaattcaatgtgattggcgaaccggaagtgttttattgcaaaccggccgatgattatctgccgcagccgggtgcggtgctgattaccggtattaccccgcaggaagcgcgcgcgaaaggtgaaaacgaagcggcgtttgccgcgcgcattcatagcctgtttaccgtgccgaaaacctgcattctgggctataacaatgtgcgcttcgatgatgaagttacccgtaatatcttttatcgtaacttttatgatccgtatgcgtggagctggcagcatgataacagccgttgggatctgctggatgtgatgcgcgcgtgctatgcgctgcgcccggaaggcattaattggccggaaaacgatgatggcctgccgagctttcgtctggaacatctgaccaaagccaacggcattgaacatagcaatgcccatgatgcgatggccgatgtttatgcgaccattgcgatggcgaaactggttaaaacccgtcagccgcgcctgtttgattatctgtttacccaccgtaacaaacacaaactgatggcgctgattgatgttccgcagatgaaaccgctggtgcatgtgagcggcatgtttggcgcctggcgcggcaacaccagctgggtggccccgctggcctggcacccggaaaatcgtaacgccgtgattatggttgatctggccggtgatattagcccgctgctggaactggatagcgataccctgcgtgaacgcctgtataccgccaaaaccgatctgggcgataatgccgccgtgccggtgaaactggttcacattaacaaatgcccggtgctggcccaggcgaacaccctgcgcccggaagatgcggatcgtctgggtattaatcgccagcattgtctggataatctgaaaatcctgcgtgaaaacccgcaggtgcgtgaaaaagtggtggcgatcttcgcggaagcggaaccgttcaccccgagcgataacgtggatgcgcagctgtataacggcttctttagcgatgccgatcgcgcggcgatgaaaatcgttctggaaaccgaaccgcgcaatctgccggcgctggatattacctttgttgataaacgtattgaaaaactgctgtttaattatcgtgcgcgcaattttccgggtaccctggattatgccgaacagcagcgttggctggaacatcgtcgtcaggttttcaccccggaatttctgcagggttatgcggatgaactgcagatgctggttcagcagtatgccgatgataaagaaaaagtggcgctgctgaaagcgctgtggcagtatgcggaagaaatcgtttctggctctggttccggcagcggttccggaacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtataggagtactttaacttatggattcaacggtaatgttactggtgatgatacaggaaaaattggcggctgtattggtgcacaagtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaatggtggttcgggctcatctggtggctcgagtcaccatcatcatcaccac

SEQ ID NO: 22 - protein, 804 aa, synthetic construct
  REGION 1..804: note = Synthetic Construct
ADSDINIKTGTTDIGSNTSGSGSGSGSGMMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGSGSGSGTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYRSTLTYGFNGNVTGDDTGKIGGCIGAQVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGGSGSSGGSSHHHHHH

SEQ ID NO: 23 - DNA, 2265 bp, synthetic construct
  misc_feature 1..2265: note = DNA Sequence encoding HL-RQC-TthRecJ-L1-H6
  source 1..2265: mol_type = other DNA
  CDS 4..2265
atggcagattctgatattaatattaaaaccggtactacagatattggaagcaatacttccggaagcggctctggtagtggttctggcatgtttcgtcgtaaagaagatctggatccgccgctggcactgctgccgctgaaaggcctgcgcgaagccgccgcactgctggaagaagcgctgcgtcaaggtaaacgcattcgtgttcacggcgactatgatgcggatggcctgaccggcaccgcgatcctggttcgtggtctggccgccctgggtgcggatgttcatccgtttatcccgcaccgcctggaagaaggctatggtgtcctgatggaacgcgtcccggaacatctggaagcctcggacctgtttctgaccgttgactgcggcattaccaaccatgcggaactgcgcgaactgctggaaaatggcgtggaagtcattgttaccgatcatcatacgccgggcaaaacgccgccgccgggtctggtcgtgcatccggcgctgacgccggatctgaaagaaaaaccgaccggcgcaggcgtggcgtttctgctgctgtgggcactgcatgaacgcctgggcctgccgccgccgctggaatacgcggacctggcagccgttggcaccattgccgacgttgccccgctgtggggttggaatcgtgcactggtgaaagaaggtctggcacgcatcccggcttcatcttgggtgggcctgcgtctgctggctgaagccgtgggctataccggcaaagcggtcgaagtcgctttccgcatcgcgccgcgcatcaatgcggcttcccgcctgggcgaagcggaaaaagccctgcgcctgctgctgacggatgatgcggcagaagctcaggcgctggtcggcgaactgcaccgtctgaacgcccgtcgtcagaccctggaagaagcgatgctgcgcaaactgctgccgcaggccgacccggaagcgaaagccatcgttctgctggacccggaaggccatccgggtgttatgggtattgtggcctctcgcatcctggaagcgaccctgcgcccggtctttctggtggcccagggcaaaggcaccgtgcgttcgctggctccgatttccgccgtcgaagcactgcgcagcgcggaagatctgctgctgcgttatggtggtcataaagaagcggcgggtttcgcaatggatgaagcgctgtttccggcgttcaaagcacgcgttgaagcgtatgccgcacgtttcccggatccggttcgtgaagtggcactgctggatctgctgccggaaccgggcctgctgccgcaggtgttccgtgaactggcactgctggaaccgtatggtgaaggtaacccggaaccgctgttcctgtctggctctggttccggcagcggttccggaacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtataggagtactttaacttatggattcaacggtaatgttactggtgatgatacaggaaaaattggcggctgtattggtgcacaagtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaatggtggttcgggctcatctggtggctcgagtcaccatcatcatcaccac

SEQ ID NO: 24 - protein, 754 aa, synthetic construct
  REGION 1..754: note = Synthetic Construct
ADSDINIKTGTTDIGSNTSGSGSGSGSGMFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFLSGSGSGSGSGTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYRSTLTYGFNGNVTGDDTGKIGGCIGAQVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGGSGSSGGSSHHHHHH

SEQ ID NO: 25 - DNA, 1785 bp, synthetic construct
  misc_feature 1..1785: note = DNA Sequence encoding HL-RQC-EcoExoIII-L2-D45-N47delta-H6
  source 1..1785: mol_type = other DNA
  CDS 4..1785
atggcagattctgatattaatattaaaaccggtactacagatattggaagcaatactacagtaaaaacaggtgatttagtcacttatgataaagaaaatggcatgcacaaaaaagtattttatagttttatcgattccggaagcggctctggtagtggttctggcatgaaatttgttagcttcaatatcaacggcctgcgcgcgcgcccgcatcagctggaagcgattgtggaaaaacatcagccggatgttattggtctgcaggaaaccaaagttcacgatgatatgtttccgctggaagaagtggcgaaactgggctataacgtgttttatcatggccagaaaggtcattatggcgtggccctgctgaccaaagaaaccccgatcgcggttcgtcgtggttttccgggtgatgatgaagaagcgcagcgtcgtattattatggcggaaattccgagcctgctgggcaatgtgaccgttattaacggctattttccgcagggcgaaagccgtgatcatccgattaaatttccggccaaagcgcagttctatcagaacctgcagaactatctggaaaccgaactgaaacgtgataatccggtgctgatcatgggcgatatgaacattagcccgaccgatctggatattggcattggcgaagaaaaccgtaaacgctggctgcgtaccggtaaatgcagctttctgccggaagaacgtgaatggatggatcgcctgatgagctggggcctggtggatacctttcgtcatgcgaacccgcagaccgccgatcgctttagctggtttgattatcgcagcaaaggttttgatgataaccgtggcctgcgcattgatctgctgctggcgagccagccgctggcggaatgctgcgttgaaaccggtattgattatgaaattcgcagcatggaaaaaccgagcgatcacgccccggtgtgggcgacctttcgccgctctggctctggttccggcagcggttccggacacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtataggagtactttaacttatggattcaacggtaatgttactggtgatgatacaggaaaaattggcggctgtattggtgcacaagtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaatggtggttcgggctcatctggtggctcgagtcaccatcatcatcaccac

SEQ ID NO: 26 - protein, 594 aa, synthetic construct
  REGION 1..594: note = Synthetic Construct
ADSDINIKTGTTDIGSNTTVKTGDLVTYDKENGMHKKVFYSFIDSGSGSGSGSGMKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRRSGSGSGSGSGHNKKLLVIRTKGTIAGQYRVYSEEGANKSGLAWPSAFKVQLQLPDNEVAQISDYYPRNSIDTKEYRSTLTYGFNGNVTGDDTGKIGGCIGAQVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGGSGSSGGSSHHHHHH

SEQ ID NO: 27 - DNA, 2364 bp, synthetic construct
  misc_feature 1..2364: note = DNA Sequence encoding HL-RQC-EcoExoI-Cter-{SG}8-H6
  source 1..2364: mol_type = other DNA
  CDS 4..2364
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtataggagtactttaacttatggattcaacggtaatgttactggtgatgatacaggaaaaattggcggctgtattggtgcacaagtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattccggtagcggctctggttctggctctggttccggcagcggttccggacagagcaccttcctgtttcatgattatgaaaccttcggtacccatccggccctggatcgtccggcgcagtttgcggccattcgcaccgatagcgaattcaatgtgattggcgaaccggaagtgttttattgcaaaccggccgatgattatctgccgcagccgggtgcggtgctgattaccggtattaccccgcaggaagcgcgcgcgaaaggtgaaaacgaagcggcgtttgccgcgcgcattcatagcctgtttaccgtgccgaaaacctgcattctgggctataacaatgtgcgcttcgatgatgaagttacccgtaatatcttttatcgtaacttttatgatccgtatgcgtggagctggcagcatgataacagccgttgggatctgctggatgtgatgcgcgcgtgctatgcgctgcgcccggaaggcattaattggccggaaaacgatgatggcctgccgagctttcgtctggaacatctgaccaaagccaacggcattgaacatagcaatgcccatgatgcgatggccgatgtttatgcgaccattgcgatggcgaaactggttaaaacccgtcagccgcgcctgtttgattatctgtttacccaccgtaacaaacacaaactgatggcgctgattgatgttccgcagatgaaaccgctggtgcatgtgagcggcatgtttggcgcctggcgcggcaacaccagctgggtggccccgctggcctggcacccggaaaatcgtaacgccgtgattatggttgatctggccggtgatattagcccgctgctggaactggatagcgataccctgcgtgaacgcctgtataccgccaaaaccgatctgggcgataatgccgccgtgccggtgaaactggttcacattaacaaatgcccggtgctggcccaggcgaacaccctgcgcccggaagatgcggatcgtctgggtattaatcgccagcattgtctggataatctgaaaatcctgcgtgaaaacccgcaggtgcgtgaaaaagtggtggcgatcttcgcggaagcggaaccgttcaccccgagcgataacgtggatgcgcagctgtataacggcttctttagcgatgccgatcgcgcggcgatgaaaatcgttctggaaaccgaaccgcgcaatctgccggcgctggatattacctttgttgataaacgtattgaaaaactgctgtttaattatcgtgcgcgcaattttccgggtaccctggattatgccgaacagcagcgttggctggaacatcgtcgtcaggttttcaccccggaatttctgcagggttatgcggatgaactgcagatgctggttcagcagtatgccgatgataaagaaaaagtggcgctgctgaaagcgctgtggcagtatgcggaagaaatcgtttctggctctggtcaccatcatcatcaccac

SEQ ID NO: 28 - protein, 787 aa, synthetic construct
  REGION 1..787: note = Synthetic Construct
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYRSTLTYGFNGNVTGDDTGKIGGCIGAQVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNSGSGSGSGSGSGSGSGQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 29 - DNA, 2370 bp, synthetic construct
  misc_feature 1..2370: note = DNA Sequence encoding HL-RQC-EcoExoI-Cter-DG{SG}8-H6
  source 1..2370: mol_type = other DNA
  CDS 4..2370
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtataggagtactttaacttatggattcaacggtaatgttactggtgatgatacaggaaaaattggcggctgtattggtgcacaagtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaatgatggctccggtagcggctctggttctggctctggttccggcagcggttccggacagagcaccttcctgtttcatgattatgaaaccttcggtacccatccggccctggatcgtccggcgcagtttgcggccattcgcaccgatagcgaattcaatgtgattggcgaaccggaagtgttttattgcaaaccggccgatgattatctgccgcagccgggtgcggtgctgattaccggtattaccccgcaggaagcgcgcgcgaaaggtgaaaacgaagcggcgtttgccgcgcgcattcatagcctgtttaccgtgccgaaaacctgcattctgggctataacaatgtgcgcttcgatgatgaagttacccgtaatatcttttatcgtaacttttatgatccgtatgcgtggagctggcagcatgataacagccgttgggatctgctggatgtgatgcgcgcgtgctatgcgctgcgcccggaaggcattaattggccggaaaacgatgatggcctgccgagctttcgtctggaacatctgaccaaagccaacggcattgaacatagcaatgcccatgatgcgatggccgatgtttatgcgaccattgcgatggcgaaactggttaaaacccgtcagccgcgcctgtttgattatctgtttacccaccgtaacaaacacaaactgatggcgctgattgatgttccgcagatgaaaccgctggtgcatgtgagcggcatgtttggcgcctggcgcggcaacaccagctgggtggccccgctggcctggcacccggaaaatcgtaacgccgtgattatggttgatctggccggtgatattagcccgctgctggaactggatagcgataccctgcgtgaacgcctgtataccgccaaaaccgatctgggcgataatgccgccgtgccggtgaaactggttcacattaacaaatgcccggtgctggcccaggcgaacaccctgcgcccggaagatgcggatcgtctgggtattaatcgccagcattgtctggataatctgaaaatcctgcgtgaaaacccgcaggtgcgtgaaaaagtggtggcgatcttcgcggaagcggaaccgttcaccccgagcgataacgtggatgcgcagctgtataacggcttctttagcgatgccgatcgcgcggcgatgaaaatcgttctggaaaccgaaccgcgcaatctgccggcgctggatattacctttgttgataaacgtattgaaaaactgctgtttaattatcgtgcgcgcaattttccgggtaccctggattatgccgaacagcagcgttggctggaacatcgtcgtcaggttttcaccccggaatttctgcagggttatgcggatgaactgcagatgctggttcagcagtatgccgatgataaagaaaaagtggcgctgctgaaagcgctgtggcagtatgcggaagaaatcgtttctggctctggtcaccatcatcatcaccac

SEQ ID NO: 30 - protein, 789 aa, synthetic construct
  REGION 1..789: note = Synthetic Construct
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYRSTLTYGFNGNVTGDDTGKIGGCIGAQVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNDGSGSGSGSGSGSGSGSGQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 31 - DNA, 50 bp, synthetic construct
  misc_feature 1..50: note = DNA oligonucleotide for exonuclease assay
  source 1..50: mol_type = other DNA
gcaacagagctgatggatcaaatgcattaggtaaacatgttacgtcgtaa

SEQ ID NO: 32 - DNA, 55 bp, synthetic construct
  misc_feature 1..55: note = DNA oligonucleotide for exonuclease assay
  source 1..55: mol_type = other DNA
cgatcttacgacgtaacatgtttacctaatgcatttgatccatcagctctgttgc

SEQ ID NO: 33 - DNA, 12 bp, synthetic construct
  misc_feature 1..12: note = Synthetic polynucleotide
  source 1..12: mol_type = other DNA
ttgcaacagagc

(19) United States
(12) Patent Application Publication
(10) Pub. No.: US 2024/0240246 A1
(43) Pub. Date: Jul. 18, 2024

(54) ENZYME-PORE CONSTRUCTS

(71) Applicant: Oxford Nanopore Technologies PLC, Oxford (GB)

(72) Inventors: Lakmal Jayasinghe, Oxford (GB); John Hagan Pryce Bayley, Oxford (GB); Stephen Cheley, East Lansing, MI (US); Brian McKeown, Middle Barton Oxon (GB); James White, Oxford (GB); James Anthony Clarke, Oxford (GB)

(73) Assignee: Oxford Nanopore Technologies PLC, Oxford (GB)

(21) Appl. No.: 18/510,933

(22) Filed: Nov. 16, 2023

Related U.S. Application Data
(63) Continuation of application No. 17/355,668, filed on Jun. 23, 2021, now Pat. No. 11,859,247.
Application No. 17/355,668 is a continuation of application No. 16/129,693, filed on Sep. 12, 2018, now Pat. No. 11,078,530.
Application No. 16/129,693 is a continuation of application No. 14/858,138, filed on Sep. 18, 2015, now Pat. No. 10,077,471.
Application No. 14/858,138 is a division of application No. 14/455,394, filed on Aug. 8, 2014, now Pat. No. 9,885,078.
Application No. 14/455,394 is a division of application No. 13/002,709, filed on May 13, 2011, now abandoned.
(60) Provisional application No. 61/078,695, filed on Jul. 7, 2008.

(51) Int. Cl. C12Q 1/6869; C07K 14/31; C12N 9/12; C12N 9/16; C12N 9/22; C12N 9/52; C12N 9/90; C12N 9/96

(52) U.S. Cl. CPC C12Q 1/6869; C07K 14/31; C12N 9/1247; C12N 9/1252; C12N 9/127; C12N 9/1276; C12N 9/16; C12N 9/22; C12N 9/52; C12N 9/90; C12N 9/96

(57) ABSTRACT

The invention relates to constructs comprising a transmembrane protein pore subunit and a nucleic acid handling enzyme. The pore subunit is covalently attached to the enzyme such that both the subunit and enzyme retain their activity. The constructs can be used to generate transmembrane protein pores having a nucleic acid handling enzyme attached thereto. Such pores are particularly useful for sequencing nucleic acids. The enzyme handles the nucleic acid in such a way that the pore can detect its component nucleotides by stochastic sensing.

### RELATED APPLICATIONS

This application is a divisional of U.S. patent application Ser. No. 13/002,709, filed May 13, 2011, which is a 35 U.S.C. 371 national stage filing of International Application No. PCT/GB2009/001679 filed Jul. 6, 2009, which claims priority to U.S. Provisional Patent Application No. 61/078,695 filed Jul. 7, 2008. The contents of the aforementioned applications are hereby incorporated by reference.

### FIELD OF THE INVENTION

The invention relates to constructs comprising a transmembrane protein pore subunit and a nucleic acid handling enzyme. The pore subunit is covalently attached to the enzyme such that both the subunit and enzyme retain their activity. The constructs can be used to generate transmembrane protein pores having a nucleic acid handling enzyme attached thereto. Such pores are particularly useful for sequencing nucleic acids. The enzyme handles the nucleic acid in such a way that the pore can detect each of its component nucleotides by stochastic sensing.

### BACKGROUND OF THE INVENTION

Stochastic detection is an approach to sensing that relies on the observation of individual binding events between analyte molecules and a receptor. Stochastic sensors can be created by placing a single pore of nanometer dimensions in an insulating membrane and measuring voltage-driven ionic transport through the pore in the presence of analyte molecules. The frequency of occurrence of fluctuations in the current reveals the concentration of an analyte that binds within the pore. The identity of an analyte is revealed through its distinctive current signature, notably the duration and extent of current block (Braha, O., Walker. B., Cheley, S., Kasianowicz, J. J., Song, L., Gouaux, J. E., and Bayley, H. (1997) Chem. Biol. 4, 497-505; and Bayley, H., and Cremer, P. S. (2001) Nature 413, 226-230).

Engineered versions of the bacterial pore forming toxin α-hemolysin (α-HL) have been used for stochastic sensing of many classes of molecules (Bayley, H., and Cremer, P. S. (2001) Nature 413, 226-230; Shin, S., H., Luchian, T., Cheley, S., Braha, O., and Bayley, H. (2002) Angew.Chem.Int.Ed. 41, 3707-3709; and Guan, X., Gu, L.-Q., Cheley, S., Braha, O., and Bayley. H. (2005) ChemBioChem 6, 1875-1881). In the course of these studies, it was found that attempts to engineer α-HL to bind small organic analytes directly can prove taxing, with rare examples of success (Guan, X., Gu, L.-Q., Cheley, S., Braha, O., and Bayley, H. (2005) ChemBioChem 6, 1875-1881). Fortunately, a different strategy was discovered, which utilized non-covalently attached molecular adaptors, notably cyclodextrins (Gu. L.-Q., Braha, O., Conlan, S., Cheley, S., and Bayley, H. (1999) Nature 398, 686-690), but also cyclic peptides (Sanchez-Quesada, J., Ghadiri, M. R., Bayley, H., and Braha, O. (2000) J.Am.Chem.Soc. 122, 11758-11766) and cucurbiturils (Braha, O., Webb, J., Gu, L.-Q., Kim, K., and Bayley, H. (2005) ChemPhysChem 6, 889-892). Cyclodextrins become transiently lodged in the α-HL pore and produce a substantial but incomplete channel block. Organic analytes, which bind within the hydrophobic interiors of cyclodextrins, augment this block allowing analyte detection (Gu, L.-Q., Braha, O., Conlan, S., Cheley, S., and Bayley, H. (1999) Nature 398, 686-690).

There is currently a need for rapid and cheap DNA or RNA sequencing technologies across a wide range of applications. Existing technologies are slow and expensive mainly because they rely on amplification techniques to produce large volumes of nucleic acid and require a high quantity of specialist fluorescent chemicals for signal detection. Stochastic sensing has the potential to provide rapid and cheap DNA sequencing by reducing the quantity of nucleotide and reagents required.

### SUMMARY OF THE INVENTION

The inventors have surprisingly demonstrated that covalent attachment of a transmembrane protein pore subunit to a nucleic acid handling enzyme results in a construct that is capable of both forming a pore and handling nucleic acids. The inventors have also surprisingly demonstrated that the construct can be used to generate a transmembrane protein pore that is capable of both handling a nucleic acid and sequencing the nucleic acid via stochastic sensing. The fixed nature and close proximity of the enzyme to the pore means that a proportion of the nucleotides in a target nucleic acid will interact with the pore and affect the current flowing through the pore in a distinctive manner. As a result, transmembrane protein pores comprising such constructs are useful tools for stochastic sensing and especially for sequencing nucleic acids.

Accordingly, the invention provides a construct comprising a transmembrane protein pore subunit and a nucleic acid handling enzyme, wherein the subunit is covalently attached to the enzyme, wherein the subunit retains its ability to form a pore and wherein the enzyme retains its ability to handle nucleic acids. The invention also provides:
a polynucleotide sequence which encodes a construct of the invention;
a modified pore for use in sequencing nucleic acids, comprising at least one construct of the invention;
a kit for producing a modified pore for use in sequencing nucleic acids, comprising:
   (a) at least one construct of the invention; and
   (b) any remaining subunits needed to form a pore:
a kit for producing a modified pore for use in sequencing nucleic acids, comprising:
   (b) at least one polynucleotide of the invention; and
   (c) polynucleotide sequences encoding any remaining subunits needed to form a pore;
a method of producing a construct of the invention, comprising:
   (a) covalently attaching a nucleic acid handling enzyme to a transmembrane protein pore subunit; and
   (b) determining whether or not the resulting construct is capable of forming a pore and handling nucleic acids;
a method of producing a modified pore of the invention, comprising:
   (a) covalently attaching a nucleic acid handling enzyme to a transmembrane protein pore; and
   (b) determining whether or not the resulting pore is capable of handling nucleic acids and detecting nucleotides:
method of producing a modified pore of the invention, comprising:
   (a) allowing at least one construct of the invention to form a pore with other suitable subunits; and
   (b) determining whether or not the resulting pore is capable of handling nucleic acids and detecting nucleotides.
a method of purifying a transmembrane pore comprising at least one construct of the invention, comprising:
   (a) providing the at least one construct and the other subunits required to form the pore;
   (b) oligomerising the at least one construct and other subunits on synthetic lipid vesicles; and
   (c) contacting the vesicles with a non-ionic surfactant; and
   (d) recovering the oligomerised pore;
a method of sequencing a target nucleic acid sequence, comprising:
   (a) contacting the target sequence with a pore of the invention, which comprises an exonuclease, such that the exonuclease digests an individual nucleotide from one end of the target sequence;
   (b) contacting the nucleotide with the pore so that the nucleotide interacts with the adaptor;
   (c) measuring the current passing through the pore during the interaction and thereby determining the identity of the nucleotide; and
   (d) repeating steps (a) to (c) at the same end of the target sequence and thereby determining the sequence of the target sequence; and
a method of sequencing a target nucleic acid sequence, comprising:
   (a) contacting the target sequence with a pore of the invention so that the enzyme pushes or pulls the target sequence through the pore and a proportion of the nucleotides in the target sequence interacts with the pore; and
   (b) measuring the current passing through the pore during each interaction and thereby determining the sequence of the target sequence.

### DESCRIPTION OF THE FIGURES

FIG. 1 shows how exonuclease enzymes catalyse the hydrolysis of phosphodiester bonds. Within the active site of the exonuclease, a water molecule is enabled to react with the phosphate of the 3′ end of the polynucleotide (DNA). Cleavage of the bond between the phosphate and the sugar towards the 5′ end releases a monophosphate (deoxy)nucleoside.

FIG. 2 shows the crystal structures of exonucleases used in the Example, N and C-terminus and active sites are shown for each. i) Adapted form of EcoExoIII; ii) EcoExoI; iii) TthRecJ-cd; and iv) Lambda exo.

FIG. 3 shows a cartoon of an exonuclease equipped α-HL pore. The exonuclease is genetically fused to one of the seven monomers of the heptamer, with linker arms sufficiently long to enable correct protein folding of the exonuclease moiety and the α-HL moiety.

FIG. 4 shows generic image of the protein construct generated shows the BspEI insertion point(s) in the α-HL gene. Ligation AfuExoIII, bounded by two stretches of DNA encoding a (serine/glycine)×5 repeat (shown hatched) generates a fusion protein in which a 64.5 kDa protein will be generated, under the transcriptional control of the T7 promoter shown.

FIG. 5 shows the oligomerisation of α-HL Loop 1 fusion constructs with wild-type α-HL at different protein ratios. i) HL-wt-EcoExoIII-L1-H6; ii) HL-RQC-EcoExoI-L1-H6; and iii) HL-RQC-TthRecJ-L1-H6.

FIG. 6 shows the control of homo and heteroheptamer generation by different monomer ratios. HL-RQ subunits are shown in white and fusion subunits in black. Increasing the ratio of fusion subunits to wild-type subunits increases the generation of 2:5, 1:6 and 0:7 hetero and homo-heptamers. Similarly increasing the concentration of HL-RQ monomer increases the generation of 6:1 and 5:2 heteroheptamers.

FIG. 7 shows the oligomerisation of HL-RQC-EcoExoIII-L1-H6 fusion proteins that contain a stiff polyproline EcoExoIII C-terminus linker. IVTT expressed proteins mixed in a 5:1 wild-type to fusion protein ratio in the presence of purified rabbit red blood cell membranes. i) HL-RQC-EcoExoIII-L1-{SG}5+{SG}5-H6; ii) HL-RQC-EcoExoIII-L1-{SG}5+5P-H6; iii) HL-RQC-EcoExoIII-L1-4SG+5P-H6; and iv) HL monomers.

FIG. 8 shows the Loop 2 region of a single α-hemolysin subunit with the mature heptamer. Subunit 1 shown in white, subunits 2-7 shown in grey and the loop 2 region of subunit 1 shown in black.

FIG. 9 shows the oligomerisation of alternative Loop 2 EcoExoIII fusion proteins. i) HL-(RQ)₇; ii) HL-(RQ)₆(RQC-EcoExoIII-L2a-H6)₁; iii) HL-(RQ)₆(RQC-EcoExoIII-L2a-8P-H6)₁; iv) HL-(RQ)₆(RQC-EoExoIII-L2-H48Δ-H6)₁; v) HL-(RQ)₆(RQC-EcoExoIII-L2-D45Δ-H6)₁; vi) HL-(RQ)₆(RQC-EcoExoIII-L2-D45-K46Δ-H6)₁; and vii) HL-(RQ)₆(RQC-EcoExoIII-L2-D45-N47Δ-H6)₁.

FIG. 10 shows the oligomerisation of alternative Loop 2 EcoExoIII fusion proteins. i) HL-(RQ)₇; ii) HL-(RQ)₆(RQC-EcoExoIII-L2a-H6)₁; iii) HL-(RQ)₆(RQC-EcoExoIII-L2-D45-N47Δ-H6)₁; iv) HL-(RQ)₆(RQC-EcoExoIII-L2-D46-K56Δ-H6)₁; v) HL-(RQ)₆(RQC-EcoExoIII-L2-D46Δ-H6)₁; vi) HL-(RQ)₆(RQC-EcoExoIII-L2-D46-N47Δ-H6)₁; vii) HL-(RQ)₆(RQC-EcoExoIII-L2-A1-S16Δ/D46-N47Δ-H6)₁; viii) HL-(RQ)₆(RQC-EcoExoIII-L2-F42-D46Δ-H6)₁; and ix) HL-(RQ)₆(RQC-EcoExoIII-L2-I43-D46Δ-H6)₁.

FIG. 11 shows the oligomerisation of EcoExoI C-terminus fusion proteins. a) denotes both hemolysin and enzyme-fusion protein monomers are radiolabelled, b) denotes only the fusion protein monomer is radiolabelled. i) HL-(RQ)₆(RQC-EcoExoI-Cter-{SG}8-H6)₁; ii) HL-(RQ)₆(RQC-EcoExoI-Cter-DG{SG}8-H6)₁; iii) HL-(RQ)₆(RQC-EcoExoI-Cter-WPV{SG}8-H6)₁; iv) HL-(RQ)₆(RQC-EcoExoI-Cter-DGS{P}12-H6)₁; and v) HL-(RQ)₆(RQC-EcoExoI-Cter-WPV{P}12-H6)₁.

FIGS. 12A and 12B show the effect of different surfactants on EcoExoIII activity. Bottom graph (FIG. 12B)—Sodium dodecyl sulphate (SDS): a; 0%, b; 0.1% c; 0.5%. Top graph (FIG. 12A)—n-Dodecyl-D-maltopyranoside (DDM): a; 0% k, b; 0.1%, c; 0.25%, d; 0.5%.

FIG. 13 shows the oligomerisation of E. coli BL21 (DE3) pLysS expressed α-hemolysin monomers for formation and purification of preferentially 6:1 heteroheptamers. His-tag purification is used to select between heteroheptamers and wild-type homoheptamer to give a large excess of 6:1 heteroheptamer.

FIG. 14 shows the exonuclease activity of monomer and heteroheptamer fusion proteins. Left graph—Activity of Wild-type and fusion monomers: a, 10^{-′2} dilution HL-RQC-EcoExoIII-L1-H6; b, 10^{-′4} dilution HL-RQC-EcoExoIII-L1-H6; c, 10^{-′6} dilution HL-RQC-EcoExoIII-L1-H6; d, 10^{-′2} dilution HL-RQ. Right graph—Activity of HL-(RQ)₆(RQC-EcoExoIII-L1-H6)₁: a, DDM crude extract; b, Ni-NTA purified; c, Ni-NTA purified and buffer exchange.

FIG. 15 shows base detection by the HL-(RQ)₆(RQC-EcoExoIII-L2-D46-N47Δ-H6)₁ heteroheptamer. The top trace was obtained from a heteroheptamer with a covalently attached am₆-amPDP₁-βCD adapter molecule. Further blocking events can be seen and ascribed to individual mono-phosphate nucleosides for base discrimination. The bottom graph shows the corresponding histograms of dNMP events from the top trace. Peaks, from left to right, correspond to G, T, A, C respectively. Data acquired at 400/400 mM KCl, 180 mV and 10 μM dNMPs.

### DESCRIPTION OF THE SEQUENCE LISTING

SEQ ID NO: 1 shows the polynucleotide sequence encoding one subunit of wild-type α-hemolysin (α-HL).
SEQ ID NO: 2 shows the amino acid sequence of one subunit of wild-type α-HL Amino acids 2 to 6, 73 to 75, 207 to 209, 214 to 216 and 219 to 222 form α-helices. Amino acids 22 to 30, 35 to 44, 52 to 62, 67 to 71, 76 to 91, 98 to 103, 112 to 123, 137 to 148, 154 to 159, 165 to 172, 229 to 235, 243 to 261, 266 to 271, 285 to 286 and 291 to 293 form β-strands. All the other non-terminal amino acids, namely 7 to 21, 31 to 34, 45 to 51, 63 to 66, 72, 92 to 97, 104 to 111, 124 to 136, 149 to 153, 160 to 164, 173 to 206, 210 to 213, 217, 218, 223 to 228, 236 to 242, 262 to 265, 272 to 274 and 287 to 290 form loop regions. Amino acids 1 and 294 are terminal amino acids.
SEQ ID NO: 3 shows the polynucleotide sequence encoding one subunit of α-HL M113R/N139Q (HL-RQ).
SEQ ID NO: 4 shows the amino acid sequence of one subunit of α-HL M113R/NI 39Q (HL-RQ). The same amino acids that form α-helices, β-strands and loop regions in wild-type α-HL form the corresponding regions in this subunit.
SEQ ID NO: 5 shows the pT7 α-HL BspEI knockout polynucleotide sequence (pT7-SCL_BspEI-KO). The α-HL encoding sequence is between nucleotides 2709 and 3593. The BspEI remnant is at nucleotides 3781 and 3782.
SEQ ID NO: 6 shows the polynucleotide sequence encoding one subunit of wild-type α-hemolysin containing a BspEI cloning site at position 1 (L1).
SEQ ID NO: 7 shows the polynucleotide sequence encoding one subunit of wild-type α-hemolysin containing a BspEI cloning site at position 2 (L2a).
SEQ ID NO: 8 shows the polynucleotide sequence encoding one subunit of wild-type α-hemolysin containing a BspEI cloning site at position 2 (L2b).
SEQ ID NO: 9 shows the codon optimized polynucleotide sequence derived from the xthA gene from E. coli. It encodes the exonuclease III enzyme from E. coli.
SEQ ID NO: 10 shows the amino acid sequence of the exonuclease III enzyme from E. coli. This enzyme performs distributive digestion of 5′ monophosphate nucleosides from one strand of double stranded DNA (dsDNA) in a 3′-5′ direction. Enzyme initiation on a strand requires a 5′ overhang of approximately 4 nucleotides. Amino acids 11 to 13, 15 to 25, 39 to 41, 44 to 49, 85 to 89, 121 to 139, 158 to 160, 165 to 174, 181 to 194, 198 to 202, 219 to 222, 235 to 240 and 248 to 252 form α-helices. Amino acids 2 to 7, 29 to 33, 53 to 57, 65 to 70, 75 to 78, 91 to 98, 101 to 109, 146 to 151, 195 to 197, 229 to 234 and 241 to 246 form β-strands. All the other non-terminal amino acids, 8 to 10, 26 to 28, 34 to 38, 42, 43, 50 to 52, 58 to 64, 71 to 74, 79 to 84, 90, 99, 100, 110 to 120, 140 to 145, 152 to 157, 161 to 164, 175 to 180, 203 to 218, 223 to 228, 247 and 253 to 261, form loops. Amino acids 1, 267 and 268 are terminal amino acids. The enzyme active site is formed by loop regions connecting β₁-α₁, β₃-β₄, β₅-β₆, β_{III}-α_{I}, β_{IV}-α_{II} and β_{V}-β_{VI} (consisting of amino acids 8-10, 58-64, 90, 110-120, 152-164, 175-180, 223-228 and 253-261 respectively). A single divalent metal ion is bound at residue E34 and aids nucleophilic attack on the phosphodiester bond by the D229 and H259 histidine-aspartate catalytic pair.
SEQ ID NO: 11 shows the codon optimized polynucleotide sequence derived from the sbcB gene from E. coli. It encodes the exonuclease I enzyme (EcoExoI) from E. coli.
SEQ ID NO: 12 shows the amino acid sequence of exonuclease I enzyme (EcoExoI) from E. coli. This enzyme performs processive digestion of 5′ monophosphate nucleosides from single stranded DNA (ssDNA) in a 3′-5′ direction. Enzyme initiation on a strand requires at least 12 nucleotides. Amino acids 60 to 68, 70 to 78, 80 to 93, 107 to 119, 124 to 128, 137 to 148, 165 to 172, 182 to 211, 213 to 221,234 to 241,268 to 286,313 to 324, 326 to 352, 362 to 370, 373 to 391, 401 to 454 and 457 to 475 form α-helices. Amino acids 10 to 18, 28 to 26, 47 to 50, 97 to 101, 133 to 136, 229 to 232, 243 to 251, 258 to 263, 298 to 302 and 308 to 311 form β-strands. All the other non-terminal amino acids, 19 to 27, 37 to 46, 51 to 59, 69, 79, 94 to 96, 102 to 106, 120 to 123, 129 to 132, 149 to 164, 173 to 181, 212, 222 to 228, 233, 242, 252 to 257, 264 to 267, 287 to 297, 303 to 307, 312, 325, 353 to 361, 371, 372, 392 to 400, 455 and 456, form loops. Amino acids 1 to 9 are terminal amino acids. The overall fold of the enzyme is such that three regions combine to form a molecule with the appearance of the letter C, although residues 355-358, disordered in the crystal structure, effectively convert this C into an O-like shape. The amino terminus (1-206) forms the exonuclease domain and has homology to the DnaQ superfamily, the following residues (202-354) form an SH3-like domain and the carboxyl domain (359-475) extends the exonuclease domain to form the C-like shape of the molecule. Four acidic residues of EcoExoI are conserved with the active site residues of the DnaQ superfamily (corresponding to D15, E17, D108 and D186). It is suggested a single metal ion is bound by residues D15 and 108. Hydrolysis of DNA is likely catalyzed by attack of the scissile phosphate with an activated water molecule, with H181 being the catalytic residue and aligning the nucleotide substrate.
SEQ ID NO: 13 shows the codon optimized polynucleotide sequence derived from the recJ gene from T. thermophilus. It encodes the RecJ enzyme from T. thermophilus (TthRecJ-cd).
SEQ ID NO: 14 shows the amino acid sequence of the RecJ enzyme from T. thermophilus (TthRecJ-cd). This enzyme performs processive digestion of 5′ monophosphate nucleosides from ssDNA in a 5′-3′ direction. Enzyme initiation on a strand requires at least 4 nucleotides. Amino acids 19 to 33, 44 to 61, 80 to 89, 103 to 111, 136 to 140, 148 to 163, 169 to 183, 189 to 202, 207 to 217, 223 to 240, 242 to 252, 254 to 287, 302 to 318, 338 to 350 and 365 to 382 form α-helices. Amino acids 36 to 40, 64 to 68, 93 to 96, 116 to 120, 133 to 135, 294 to 297, 321 to 325, 328 to 332, 352 to 355 and 359 to 363 form β-strands. All the other non-terminal amino acids, 34, 35, 41 to 43, 62, 63, 69 to 79, 90 to 92, 97 to 102, 112 to 115, 121 to 132, 141 to 147, 164 to 168, 184 to 188, 203 to 206, 218 to 222, 241, 253, 288 to 293, 298 to 301, 319, 320, 326, 327, 333 to 337, 351 to 358 and 364, form loops. Amino acids 1 to 18 and 383 to 425 are terminal amino acids. The crystal structure has only been resolved for the core domain of RecJ from Thermus thermophilus (residues 40-463). To ensure initiation of translation and in vivo expression of the RecJ core domain a methionine residue was added at its amino terminus, this is absent from the crystal structure information. The resolved structure shows two domains, an amino (2-253) and a carboxyl (288-463) region, connected by a long α-helix (254-287). The catalytic residues (D46, D98, H122, and D183) co-ordinate a single divalent metal ion for nucleophilic attack on the phosphodiester bond. D46 and H120 proposed to be the catalytic pair, however, mutation of any of these conserved residues in the E. coli RecJ was shown to abolish activity.
SEQ ID NO: 15 shows the codon optimized polynucleotide sequence derived from the bacteriphage lambda exo (redX) gene. It encodes the bacteriphage lambda exonuclease.
SEQ ID NO: 16 shows the amino acid sequence of the bacteriphage lambda exonuclease. The sequence is one of three identical subunits that assemble into a trimer. The enzyme performs highly processive digestion of nucleotides from one strand of dsDNA, in a 3′-5′ direction. Enzyme initiation on a strand preferentially requires a 5′ overhang of approximately 4 nucleotides with a 5′ phosphate. Amino acids 3 to 10, 14 to 16, 22 to 26, 34 to 40, 52 to 67, 75 to 95, 135 to 149, 152 to 165 and 193 to 216 form α-helices. Amino acids 100 to 101, 106 to 107, 114 to 116, 120 to 122, 127 to 131, 169 to 175 and 184 to 190 form β-strands. All the other non-terminal amino acids, 11 to 13, 17 to 21, 27 to 33, 41 to 51, 68 to 74, 96 to 99, 102 to 105, 108 to 113, 117 to 119, 123 to 126, 132 to 134, 150 to 151, 166 to 168, 176 to 183, 191 to 192, 217 to 222, form loops. Amino acids 1, 2 and 226 are terminal amino acids. Lambda exonuclease is a homo-trimer that forms a toroid with a tapered channel through the middle, apparently large enough for dsDNA to enter at one end and only ssDNA to exit at the other. The catalytic residues are undetermined but a single divalent metal ion appears bound at each subunit by residues D119, E129 and L130.
SEQ ID NO: 17 shows the polynucleotide sequence encoding HL-wt-EcoExoIII-L1-H6 used in the Example.
SEQ ID NO: 18 shows the amino acid sequence of one subunit of HL-wt-EcoExoIII-L1-H6 used in the Example.
SEQ ID NO: 19 shows the polynucleotide sequence encoding HL-RQC-EcoExoIII-L1-H6 used in the Example.
SEQ ID NO: 20 shows the amino acid sequence of one subunit of HL-RQC-EcoExoIII-L1-H6 used in the Example.
SEQ ID NO: 21 shows the polynucleotide sequence encoding HL-RQC-EcoExoI-L1-H6 used in the Example.
SEQ ID NO: 22 shows the amino acid sequence of one subunit of HL-RQC-EcoExoI-L1-H6 used in the Example.
SEQ ID NO: 23 shows the polynucleotide sequence encoding HL-RQC-TthRecJ-L1-H6 used in the Example.
SEQ ID NO: 24 shows the amino acid sequence of one subunit of HL-RQC-TthRecJ-L1-H6 used in the Example.
SEQ ID NO: 25 shows the polynucleotide sequence encoding HL-RQC-EcoExoIII-L2-D45-N47Δ-H6 used in the Example.
SEQ ID NO: 26 shows the amino acid sequence of one subunit of HL-RQC-EcoExoIII-L2-D45-N47Δ-H6 used in the Example.
SEQ ID NO: 27 shows the polynucleotide sequence encoding HL-RQC-EcoExoI-Cter-{SG}8-H6 used in the Example.
SEQ ID NO: 28 shows the amino acid sequence of one subunit of HL-RQC-EcoExoI-Cter-{SG}8-H6 used in the Example.
SEQ ID NO: 29 shows the polynucleotide sequence encoding HL-RQC-EcoExoI-Cter-DG{SG}8-H6 used in the Example.
SEQ ID NO: 30 shows the amino acid sequence of one subunit of HL-RQC-EcoExoI-Cter-DG{SG}8-H6 used in the Example.
SEQ ID NOs: 31 and 32 show the oligonucleotide sequences used in the exonuclease assay of the Example.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a” “an”, and “the” include plural referents unless the content clearly dictates otherwise. Thus, for example, reference to “a construct” includes “constructs”, reference to “a transmembrane protein pore” includes two or more such pores, reference to “a molecular adaptor” includes two or more such adaptors, and the like.

All publications, patents and patent applications cited herein, whether supra or infra, are hereby incorporated by reference in their entirety.

### Constructs

The present invention provides constructs that are useful for sequencing nucleic acids. The constructs comprise a transmembrane protein pore subunit and a nucleic acid handling enzyme. The subunit is covalently attached to the enzyme. The constructs of the invention are useful tools for forming pores that are capable of sequencing nucleic acids by stochastic sensing. The constructs of the invention are particularly useful for generating transmembrane protein pores that can both handle a target nucleic acid sequence and discriminate between the different nucleotides in the target sequence. As described in more detail below, the enzyme handles a target nucleic acid in such a way that the pore can identify nucleotides in the target sequence and thereby sequence the target sequence.

The subunit retains its ability to form a pore. The ability of a construct to form a pore can be assayed using any method known in the art. For instance, the construct may be inserted into a membrane along with other appropriate subunits and its ability to oligomerize to form a pore may be determined. Methods are known in the art for inserting constructs and subunits into membranes, such as lipid bilayers. For example, constructs and subunits may be suspended in a purified form in a solution containing a lipid bilayer such that it diffuses to the lipid bilayer and is inserted by binding to the lipid bilayer and assembling into a functional state. Alternatively, constructs and subunits may be directly inserted into the membrane using the “pick and place” method described in M. A. Holden, H. Bayley. J. Am. Chem. Soc. 2005, 127, 6502-6503 and International Application No. PCT/GB2006/001057 (published as WO 2006/100484). The ability of a construct to form a pore is typically assayed as described in the Examples.

The enzyme retains its ability to handle nucleic acids. This allows the construct to form a pore that may be used to sequence nucleic acids as described below. The ability of a construct to handle nucleic acids can be assayed using any method known in the art. For instance, construct or pores formed from the constructs can be tested for their ability to handle specific sequences of nucleic acids. The ability of a construct or a pore to handle nucleic acids is typically assayed as described in the Examples.

A construct of the invention may form part of a pore. Alternatively, a construct may be isolated, substantially isolated, purified or substantially purified. A construct is isolated or purified if it is completely free of any other components, such as lipids or other pore monomers. A construct is substantially isolated if it is mixed with carriers or diluents which will not interfere with its intended use. For instance, a construct is substantially isolated or substantially purified if it present in a form that comprises less than 10%, less than 5%, less than 2% or less than 1% of other components, such as lipids or other pore monomers. A construct of the invention may be present in a lipid bilayer.

### Attachment

The subunit is covalently attached to the enzyme. The subunit may be attached to the enzyme at more than one, such as two or three, points. Attaching the subunit to the enzyme at mom than one point can be used to constrain the mobility of the enzyme. For instance, multiple attachments may be used to constrain the freedom of the enzyme to rotate or its ability to move away from the subunit.

The subunit may be in a monomeric form when it is attached to the enzyme (post expression modification). Alternatively, the subunit may be part of an oligomeric pore when it is attached to an enzyme (post oligomerisation modification).

The subunit can be covalently attached to the enzyme using any method known in the art. The subunit and enzyme may be produced separately and then attached together. The two components may be attached in any configuration. For instance, they may be attached via their terminal (i.e. amino or carboxy terminal) amino acids. Suitable configurations include, but are not limited to, the amino terminus of the enzyme being attached to the carboxy terminus of the subunit and vice versa. Alternatively, the two components may be attached via amino acids within their sequences. For instance, the enzyme may be attached to one or more amino acids in a loop region of the subunit. In a preferred embodiment, terminal amino acids of the enzyme are attached to one or more amino acids in the loop region of a subunit. Terminal amino acids and loop regions are discussed above.

In one preferred embodiment, the subunit is genetically fused to the enzyme. A subunit is genetically fused to an enzyme if the whole construct is expressed from a single polynucleotide sequence. The coding sequences of the subunit and enzyme may be combined in any way to form a single polynucleotide sequence encoding the construct.

The subunit and enzyme may be genetically fused in any configuration. The subunit and enzyme may be fused via their terminal amino acids. For instance, the amino terminus of the enzyme may be fused to the carboxy terminus of the subunit and vice versa. The amino acid sequence of the enzyme is preferably added in frame into the amino acid sequence of the subunit. In other words, the enzyme is preferably inserted within the sequence of the subunit. In such embodiments, the subunit and enzyme are typically attached at two points, i.e. via the amino and carboxy terminal amino acids of the enzyme. If the enzyme is inserted within the sequence of the subunit, it is preferred that the amino and carboxy terminal amino acids of the enzyme are in close proximity and are each attached to adjacent amino acids in the sequence of the subunit or variant thereof. In a preferred embodiment, the enzyme is inserted into a loop region of the subunit.

In another preferred embodiment, the subunit is chemically fused to the enzyme. A subunit is chemically fused to an enzyme if the two parts are chemically attached, for instance via a linker molecule.

The subunit may be transiently attached to the enzyme by a hex-his tag or Ni-NTA. The subunit and enzyme may also be modified such that they transiently attach to each other.

The construct retains the pore forming ability of the subunit. The pore forming ability of the subunit is typically provided by its α-helices and β-strands. β-barrel pores comprise a barrel or channel that is formed from β-strands, whereas α-helix bundle pores comprise a barrel or channel that is formed from α-helices. The α-helices and D-strands are typically connected by loop regions. In order to avoid affecting the pore forming ability of the subunit, the enzyme is preferably genetically fused to a loop region of the subunit or inserted into a loop region of the subunit. The loop regions of specific subunits are discussed in more detail below.

Similarly, the construct retains the nucleic acid handling ability of the enzyme, which is also typically provided by its secondary structural elements (α-helices and β-strands) and tertiary structural elements. In order to avoid affecting the nucleic acid handling ability of the enzyme, the enzyme is preferably genetically fused to the subunit or inserted into the subunit via residues or regions that does not affect its secondary or tertiary structure.

The subunit may be attached directly to the enzyme. The subunit is preferably attached to the enzyme using one or more, such as two or three, linkers. The one or more linkers may be designed to constrain the mobility of the enzyme. The linkers may be attached to one or more reactive cysteine residues, reactive lysine residues or non-natural amino acids in the subunit and/or enzyme. Suitable linkers are well-known in the art. Suitable linkers include, but are not limited to, chemical crosslinkers and peptide linkers. Preferred linkers are amino acid sequences (i.e. peptide linkers). The length, flexibility and hydrophilicity of the peptide linker are typically designed such that it does not to disturb the functions of the subunit and enzyme. Preferred flexible peptide linkers are stretches of 2 to 20, such as 4, 6, 8, 10 or 16, serine and/or glycine amino acids. More preferred flexible linkers include (SG)₁, (SG)₂. (SG)₃, (SG)₄, (SG)₅ and (SG)₆ wherein S is serine and G is glycine. Preferred rigid linkers are stretches of 2 to 30, such as 4, 6, 8, 16 or 24, proline amino acids. More preferred rigid linkers include (P)₁₂ wherein P is proline.

Linkers may be attached to the subunit first and then the enzyme, the enzyme first and then the subunit or the enzyme and subunit at the same time. When the linker is attached to the subunit, it may be a monomeric subunit, part of an oligomer of two or more monomers or part of complete oligomeric pore. It is preferred that the linker is reacted before any purification step to remove any unbound linker.

A preferred method of attaching the subunit to the enzyme is via cysteine linkage. This can be mediated by a bi-functional chemical linker or by a polypeptide linker with a terminal presented cysteine residue. α-HL (SEQ ID NO: 2) lacks native cysteine residues so the introduction of a cysteine into the sequence of SEQ ID NO: 2 enables the controlled covalent attachment of the enzyme to the subunit. Cysteines can be introduced at various positions, such as position K8, T9 or N17 of SEQ ID NO: 2 or at the carboxy terminus of SEQ ID NO: 2. The length, reactivity, specificity, rigidity and solubility of any bi-functional linker may designed to ensure that the enzyme is positioned correctly in relation to the subunit and the function of both the subunit and enzyme is retained. Suitable linkers include bismaleimide crosslinkers, such as 1,4-bis(maleimido)butane (BMB) or bis(maleimido)hexane. One draw back of bi-functional linkers is the requirement of the enzyme to contain no further surface accessible cysteine residues, as binding of the bi-functional linker to these cannot be controlled and may affect substrate binding or activity. If the enzyme does contain several accessible cysteine residues, modification of the enzyme may be required to remove them while ensuring the modifications do not affect the folding or activity of the enzyme. In a preferred embodiment, a reactive cysteine is presented on a peptide linker that is genetically attached to the enzyme. This means that additional modifications will not necessarily be needed to remove other accessible cysteine residues from the enzyme. The reactivity of cysteine residues may be enhanced by modification of the adjacent residues, for example on a peptide linker. For instance, the basic groups of flanking arginine, histidine or lysine residues will change the pKa of the cysteines thiol group to that of the more reactive S⁻ group. The reactivity of cysteine residues may be protected by thiol protective groups such as dTNB. These may be reacted with one or more cysteine residues of the enzyme or subunit, either as a monomer or part of an oligomer, before a linker is attached.

Cross-linkage of subunits or enzymes to themselves may be prevented by keeping the concentration of linker in a vast excess of the subunit and/or enzyme. Alternatively, a “lock and key” arrangement may be used in which two linkers are used. Only one end of each linker may react together to form a longer linker and the other ends of the linker each react with a different part of the construct (i.e. subunit or monomer).

The site of covalent attachment is selected such that, when the construct is used to form a pore, the enzyme handles a target nucleic acid sequence in such a way that a proportion of the nucleotides in the target sequence interacts with the pore. Nucleotides are then distinguished on the basis of the different ways in which they affect the current flowing through the pore during the interaction.

There are a number of ways that pores can be used to sequence nucleic acid molecules. One way involves the use of an exonuclease enzyme, such as a deoxyribonuclease. In this approach, the exonuclease enzyme is used to sequentially detach the nucleotides from a target nucleic strand. The nucleotides are then detected and discriminated by the pore in order of their release, thus reading the sequence of the original strand. For such an embodiment, the exonuclease enzyme is preferably attached to the subunit such that a proportion of the nucleotides released from the target nucleic acid is capable of entering and interacting with the barrel or channel of a pore comprising the construct. The exonuclease is preferably attached to the subunit at a site in close proximity to the part of the subunit that forms the opening of the barrel of channel of the pore. The exonuclease enzyme is more preferably attached to the subunit such that its nucleotide exit trajectory site is orientated towards the part of the subunit that forms part of the opening of the pore.

Another way of sequencing nucleic acids involves the use of an enzyme that pushes or pulls the target nucleic acid strand through the pore. In this approach, the ionic current fluctuates as a nucleotide in the target strand passes through the pore. The fluctuations in the current are indicative of the sequence of the strand. For such an embodiment, the enzyme is preferably attached to the subunit such that it is capable of pushing or pulling the target nucleic acid through the barrel or channel of a pore comprising the construct and does not interfere with the flow of ionic current through the pore. The enzyme is preferably attached to the subunit at a site in close proximity to the part of the subunit that forms part of the opening of the barrel of channel of the pore. The enzyme is more preferably attached to the subunit such that its active site is orientated towards the part of the subunit that forms part of the opening of the pore.

A third way of sequencing a nucleic acid strand is to detect the bi-products of a polymerase in close proximity to a pore detector. In this approach, nucleoside phosphates (nucleotides) are labelled so that a phosphate labelled species is released upon the addition of a polymerase to the nucleotide strand and the phosphate labelled species is detected by the pore. The phosphate species contains a specific label for each nucleotide. As nucleotides are sequentially added to the nucleic acid strand, the bi-products of the base addition are detected. The order that the phosphate labelled species are detected can be used to determine the sequence of the nucleic acid strand.

The enzyme is preferably attached to a part of the subunit that forms part of the cis side of a pore comprising the construct. In electrophysiology, the cis side is the grounded side. If a hemolysin pore is inserted correctly into an electrophysiology apparatus, the Cap region is on the cis side. It is well known that, under a positive potential, nucleotides will migrate from the cis to the trans side of pores used for stochastic sensing. Positioning the enzyme at the cis side of a pore allows it to handle the target nucleic acid such that a proportion of the nucleotides in the sequence enters the barrel or channel of the pore and interacts with it. Preferably, at least 20%, at least 40%, at least 50%, at least 80% or at least 90% of the nucleotides in the sequence enters the barrel or channel of the pore and interacts with it.

The site and method of covalent attachment is preferably selected such that mobility of the enzyme is constrained. This helps to ensure that the enzyme handles the target nucleic acid sequence in such a way that a proportion of the nucleotides in the target sequence interacts with the pore. For instance, constraining the ability of enzyme to move means that its active site can be permanently orientated towards the part of the subunit that forms part of the opening of the barrel of channel of the pore. The mobility of the enzyme may be constrained by increasing the number of points at which the enzyme is attached to the subunit and/or the use of specific linkers.

### Subunit

The constructs of the invention comprise a subunit from a transmembrane protein pore. A transmembrane protein pore is a polypeptide or a collection of polypeptides that permits ions driven by an applied potential to flow from one side of a membrane. The pore preferably permits nucleotides to flow from one side of a membrane to the other along the applied potential. The pore preferably allows a nucleic acid, such as DNA or RNA, to be pushed or pulled through the pore.

The subunit is part of a pore. The pore may be a monomer or an oligomer. The subunit preferably forms part of a pore made up of several repeating subunits, such as 6, 7 or 8 subunits. The subunit more preferably forms part of a heptameric pore. The subunit typically forms part of a barrel or channel through which the ions may flow. The subunits of the pore typically surround a central axis and contribute strands to a transmembrane β barrel or channel or a transmembrane α-helix bundle or channel. When part of a construct of the invention, the subunit may be a monomer or part of an oligomeric pore.

The subunit typically forms part of a pore whose barrel or channel comprises amino acids that facilitate interaction with nucleotides or nucleic acids. These amino acids are preferably located near the constriction of the barrel or channel. The subunit typically comprises one or more positively charged amino acids, such as arginine, lysine or histidine. These amino acids typically facilitate the interaction between the pore and nucleotides or nucleic acids by interacting with the phosphate groups in the nucleotides or nucleic acids or by n-cation interaction with the bases in the nucleotides or nucleic acids. The nucleotide detection can be facilitated with an adaptor.

Subunits for use in accordance with the invention can be derived from @-barrel pores or α-helix bundle pores. β-barrel pores comprise a barrel or channel that is formed from β-strands. Suitable β-barrel pores include, but are not limited to, β-toxins, such as α-hemolysin and leukocidins, and outer membrane proteins/porins of bacteria, such as outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and a outer membrane proteins, such as WZA.

The subunit is preferably derived from α-hemolysin (α-HL). The wild-type α-HL pore is formed of seven identical monomers or subunits (i.e. it is heptameric). The sequence of one wild-type monomer or subunit of α-hemolysin is shown in SEQ ID NO: 2. The subunit in the constructs of the invention preferably comprises the sequence shown in SEQ ID NO: 2 or a variant thereof. Amino acids 1, 7 to 21, 31 to 34, 45 to 51, 63 to 66, 72, 92 to 97, 104 to 111, 124 to 136, 149 to 153, 160 to 164, 173 to 206, 210 to 213, 217, 218, 223 to 228,236 to 242, 262 to 265, 272 to 274, 287 to 290 and 294 of SEQ ID NO: 2 form loop regions. The enzyme is preferably attached to one or more of amino acids 8, 9, 17, 18, 19, 44, 45, 50 and 51 of SEQ ID NO: 2. The enzyme is more preferably inserted between amino acids, 18 and 19, 44 and 45 or 50 and 51 of SEQ ID NO: 2.

A variant of SEQ ID NO: 2 is a subunit that has an amino acid sequence which varies from that of SEQ ID NO: 2 and which retains its pore forming ability. The ability of the variant to form pores can be assayed as described above. The variant may include modifications that facilitate covalent attachment to or interaction with the nucleic acid handling enzyme. The variant preferably comprises one or more reactive cysteine residues that facilitate attachment to the enzyme. For instance, the variant may include a cysteine at one or more of positions 8, 9, 17, 18, 19, 44, 45, 50 and 51 and/or on the amino or carboxy terminus of SEQ ID NO: 2. Preferred variants comprise a substitution of the residue at position 8, 9 or 17 of SEQ ID NO: 2 with cysteine (K8C, T9C or N17C).

The variant may be modified to facilitate genetic fusion of the enzyme. For instance, one or more residues adjacent to the insertion site may be modified, such as deleted, to facilitate insertion of the enzyme and/or linkers. If the enzyme is inserted into loop 2 of SEQ ID NO: 2, one or more of residues D45, K46, N47, H48, N49 and K50 of SEQ ID NO: 2 may be deleted. A preferred construct containing such a deletion comprises the sequence shown in SEQ ID NO: 26 or a variant thereof.

The variant may also include modifications that facilitate any interaction with nucleotides or facilitate orientation of a molecular adaptor as discussed below. The variant may also contain modifications that facilitate covalent attachment of a molecular adaptor.

The subunit may be any of the variants of SEQ ID NO: 2 described in a co-pending International application claiming priority from US Application No. 61/078,687 and being filed simultaneously with this application [J A Kemp & Co Ref: N.104403A; Oxford Nanolabs Ref: ONL IP 004]. All the teachings of that application may be applied equally to the present invention. In particular, the variant preferably has a glutamine at position 139 of SEQ ID NO: 2. The variant preferably has an arginine at position 113 of SEQ ID NO: 2. The variant preferably has a cysteine at position 119, 121 or 135 of SEQ ID NO: 2. Any of the variants of SEQ ID NO: 2 shown in SEQ ID NOs: 4, 6, 8, 10, 12 and 14 of the co-pending application may be used to form a construct of the invention.

The subunit may be a naturally occurring variant which is expressed by an organism, for instance by a Staphylococcus bacterium. Variants also include non-naturally occurring variants produced by recombinant technology. Over the entire length of the amino acid sequence of SEQ ID NO: 2, a variant will preferably be at least 50% homologous to that sequence based on amino acid identity. More preferably, the subunit polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 2 over the entire sequence. There may be at least 80%, for example at least 85%, 90% or 95%, amino acid identity over a stretch of 200 or more, for example 230, 250, 270 or 280 or more, contiguous amino acids (“hard homology”).

Amino acid substitutions may be made to the amino acid sequence of SEQ ID NO: 2 in addition to those discussed above, for example up to 1, 2, 3, 4, 5, 10, 20 or 30 substitutions. Conservative substitutions may be made, for example, according to Table 1 below.

TABLE 1
Conservative substitutions
Amino acids in the same block in the second
column and preferably in the same line in the
third column may be substituted for each other.

| | | |
|---|---|---|
| NON-AROMATIC | Non-polar | G A P |
| | | I L V |
| | Polar—uncharged | C S T M |
| | | N Q |
| | Polar—charged | D E |
| | | H K R |
| AROMATIC | | H F W Y |

One or more amino acid residues of the amino acid sequence of SEQ ID NO: 2 may additionally be deleted from the polypeptides described above. Up to 1, 2, 3, 4, 5, 10, 20 or 30 residues may be deleted, or more.

Variants may fragments of SEQ ID NO: 2. Such fragments retain pore forming activity. Fragments may be at least 50, 100, 200 or 250 amino acids in length. A fragment preferably comprises the pore forming domain of SEQ ID NO: 2. Fragments typically include residues 119, 121, 135, 113 and 139 of SEQ ID NO: 2.

One or more amino acids may be alternatively or additionally added to the polypeptides described above. An extension may be provided at the amino terminus or carboxy terminus of the amino acid sequence of SEQ ID NO: 2 or a variant or fragment thereof. The extension may be quite short, for example from 1 to 10 amino acids in length. Alternatively, the extension may be longer, for example up to 50 or 100 amino acids. A carrier protein may be fused to a subunit or variant.

As discussed above, a variant of SEQ ID NO: 2 is a subunit that has an amino acid sequence which varies from that of SEQ ID NO: 2 and which retains its ability to form a pore. A variant typically contains the regions of SEQ ID NO: 2 that are responsible for pore formation. The pore forming ability of α-HL, which contains a β-barrel, is provided by β-strands in each subunit. A variant of SEQ ID NO: 2 typically comprises the regions in SEQ ID NO: 2 that form β-strands. The amino acids of SEQ ID NO: 2 that form D-strands are discussed above. One or more modifications can be made to the regions of SEQ ID NO: 2 that form β-strands as long as the resulting variant retains its ability to form a pore. Specific modifications that can be made to the β-strand regions of SEQ ID NO: 2 are discussed above.

A variant of SEQ ID NO: 2 preferably includes one or more modifications, such as substitutions, additions or deletions, within its α-helices and/or loop regions. Amino acids that form α-helices and loops are discussed above.

Standard methods in the art may be used to determine homology. For example the UWGCG Package provides the BESTFIT program which can be used to calculate homology, for example used on its default settings (Devereux et al (1984) Nucleic Acids Research 12, p387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et at (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et at, supra). These initial neighbourhood word hits act as seeds for initiating searches to find HSP's containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The variant may be modified for example by the addition of histidine or aspartic acid residues to assist its identification or purification or by the addition of a signal sequence to promote their secretion from a cell where the polypeptide does not naturally contain such a sequence.

The subunit may be labelled with a revealing label. The revealing label may be any suitable label which allows the pore to be detected. Suitable labels include, but are not limited to, fluorescent molecules, radioisotopes, e.g. ¹²⁵I, ³⁵S, enzymes, antibodies, antigens, polynucleotides and ligands such as biotin.

The subunit may be isolated from a pore producing organism, such as Staphylococcus aureus, or made synthetically or by recombinant means. For example, the subunit may be synthesized by in vitro translation and transcription. The amino acid sequence of the subunit may be modified to include non-naturally occurring amino acids or to increase the stability of the subunit. When the subunit is produced by synthetic means, such amino acids may be introduced during production. The subunit may also be altered following either synthetic or recombinant production.

The subunit may also be produced using D-amino acids. For instance, the pores may comprise a mixture of L-amino acids and D-amino acids. This is conventional in the art for producing such proteins or peptides.

The subunit may also contain other non-specific chemical modifications as long as they do not interfere with its ability to form a pore. A number of non-specific side chain modifications are known in the art and may be made to the side chains of the pores. Such modifications include, for example, reductive alkylation of amino acids by reaction with an aldehyde followed by reduction with NaBH₄, amidination with methylacetimidate or acylation with acetic anhydride. The modifications to the subunit can be made after expression of the subunit or construct or after the subunit has been used to form a pore.

The subunit can be produced using standard methods known in the art. Polynucleotide sequences encoding a subunit may be isolated and replicated using standard methods in the art. Such sequences are discussed in more detail below. Polynucleotide sequences encoding a subunit may be expressed in a bacterial host cell using standard techniques in the art. The subunit may be produced in a cell by in situ expression of the polypeptide from a recombinant expression vector. The expression vector optionally carries an inducible promoter to control the expression of the polypeptide.

A subunit may be produced in large scale following purification by any protein liquid chromatography system from pore producing organisms or after recombinant expression as described below. Typical protein liquid chromatography systems include FPLC, AKTA systems, the Bio-Cad system, the Bio-Rad BioLogic system and the Gilson HPLC system.

### Nucleic Acid Handling Enzyme

The constructs of the invention comprise a nucleic acid handling enzyme. A nucleic acid handling enzyme is a polypeptide that is capable of interacting with and modifying at least one property of a nucleic acid. The enzyme may modify the nucleic acid by cleaving it to form individual nucleotides or shorter chains of nucleotides, such as di- or trinucleotides. The enzyme may modify the nucleic acid by orienting it or moving it to a specific position.

A nucleic acid is a macromolecule comprising two or more nucleotides. The nucleic acid handled by the enzyme may comprise any combination of any nucleotides. The nucleotides can be naturally occurring or artificial. A nucleotide typically contains a nucleobase, a sugar and at least one phosphate group. The nucleobase is typically heterocyclic. Nucleobases include, but are not limited to, purines and pyrimidines and more specifically adenine, guanine, thymine, uracil and cytosine. The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The nucleotides are preferably selected from AMP, TMP, GMP, UMP, dAMP, dTMP, dGMP or dCMP.

The nucleic acid handled by the enzyme is preferably double stranded, such as DNA. The nucleic acid handled by the enzyme may be single stranded, such as cDNA or RNA. Enzymes that handle single stranded nucleic acids may be used to sequence double stranded DNA as long as the double stranded DNA is chemically or thermally dissociated into a single strand before it is handled by the enzyme.

It is preferred that the tertiary structure of the nucleic acid handling enzyme is known. Knowledge of the three dimensional structure of the enzyme allows modifications to be made to the enzyme to facilitate its function in the construct or pore of the invention.

The enzyme may be any size and have any structure. For instance, the enzyme may be an oligomer, such as a dimer or trimer. The enzyme is preferably a small, globular polypeptide formed from one monomer. Such enzymes are easy to handle and are less likely to interfere with the pore forming ability of the subunit, particularly if fused to or inserted into the sequence of the subunit.

The amino and carboxy terminii of the enzyme are preferably in close proximity. The amino and carboxy terminii of the enzyme are more preferably presented on same face of the enzyme. Such embodiments facilitate insertion of the enzyme into the sequence of the subunit. For instance, if the amino and carboxy terminii of the enzyme are in close proximity, each can be attached by genetic fusion to adjacent amino acids in the sequence of the subunit.

It is also preferred that the location and function of the active site of the enzyme is known. This prevents modifications being made to the active site that abolish the activity of the enzyme. It also allows the enzyme to be attached to the subunit so that the enzyme handles the target nucleic acid sequence in such a way that a proportion of the nucleotides in the target sequence interacts with the pore. It is beneficial to position the active site of the enzyme as close as possible to the part of the subunit that forms part of the opening of the barrel of channel of the pore, without the enzyme itself presenting a block to the flow of current. Knowledge of the way in which an enzyme may orient nucleic acids also allows an effective construct to be designed.

As discussed in more detail below, it may be necessary to purify the construct of the invention. It is preferred that the enzyme is capable of withstanding the conditions used to purify the construct.

The constructs of the invention are useful for forming pores. Such pores may be used to sequence nucleic acids. In order that most of the nucleotides in the target nucleic acid are correctly identified by stochastic sensing, the enzyme must handle the nucleic acid in a buffer background which is compatible with discrimination of the nucleotides. The enzyme preferably has at least residual activity in a salt concentration well above the normal physiological level, such as from 100 mM to 500 mM. The enzyme is more preferably modified to increase its activity at high salt concentrations. The enzyme may also be modified to improve its processivity, stability and shelf life.

Suitable modifications can be determined from the characterisation of nucleic acid handling enzymes from extremophiles such as halophilic, moderately halophilic bacteria, thermophilic and moderately thermophilic organisms, as well as directed evolution approaches to altering the salt tolerance, stability and temperature dependence of mesophilic or thermophilic exonucleases.

The enzyme also preferably retains at least partial activity at room temperature. This allows pores formed from the construct to sequence nucleic acids at room temperature.

The nucleic acid handling enzyme is preferably a nucleolytic enzyme. The nucleic acid handling enzyme is more preferably member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31. The nucleic acid handling enzyme is more preferably any one of the following enzymes:
3.1.11.—Exodeoxyribonucleases producing 5′-phosphomonoesters.
   3.1.11.1 Exodeoxyribonuclease I.
   3.1.11.2 Exodeoxyribonuclease III.
   3.1.11.3 Exodeoxyribonuclease (lambda-induced).
   3.1.11.4 Exodeoxyribonuclease (phage SP3-induced).
   3.1.11.5 Exodeoxyribonuclease V.
   3.1.11.6 Exodeoxyribonuclease VII.
3.1.13.—Exoribonucleases producing 5′-phosphomonoesters.
   3.1.13.1 Exoribonuclease II.
   3.1.13.2 Exoribonuclease H.
   3.1.13.3 Oligonucleotidase.
   3.1.13.4 Poly(A)-specific ribonuclease.
   3.1.13.5 Ribonuclease D.
3.1.14.—Exoribonucleases producing 3′-phosphomonoesters.
   3.1.14.1 Yeast ribonuclease.
3.1.15.—Exonucleases active with either ribo- or deoxyribonucleic acid producing 5′ phosphomonoesters
   3.1.15.1 Venom exonuclease.
3.1.16.—Exonucleases active with either ribo- or deoxyribonucleic acid producing 3′ phosphomonoesters
   3.1.16.1 Spleen exonuclease.
3.1.21.—Endodeoxyribonucleases producing 5′-phosphomonoesters.
   3.1.21.1 Deoxyribonuclease 1.
   3.1.21.2 Deoxyribonuclease IV (phage-T(4)-induced).
   3.1.21.3 Type I site-specific deoxyribonuclease.
   3.1.21.4 Type II site-specific deoxyribonuclease.
   3.1.21.5 Type III site-specific deoxyribonuclease.
   3.1.21.6 CC-preferring endodeoxyribonuclease.
   3.1.21.7 Deoxyribonuclease V.
3.1.22.—Endodeoxyribonucleases producing other than 5′-phosphomonoesters.
   3.1.22.1 Deoxyribonuclease II.
   3.1.22.2 Aspergillus deoxyribonuclease K(1).
   3.1.22.3 Transferred entry: 3.1.21.7.
   3.1.22.4 Crossover junction endodeoxyribonuclease.
   3.1.22.5 Deoxyribonuclease X.
3.1.25.—Site-specific endodeoxyribonucleases specific for altered bases.
   3.1.25.1 Deoxyribonuclease (pyrimidine dimer).
   3.1.25.2 Transferred entry: 4.2.99.18.
3.1.26.—Endoribonucleases producing 5′-phosphomonoesters.
   3.1.26.1 Physarum polycephalum ribonuclease.
   3.1.26.2 Ribonuclease alpha.
   3.1.26.3 Ribonuclease III.
   3.1.26.4 Ribonuclease H.
   3.1.26.5 Ribonuclease P.
   3.1.26.6 Ribonuclease IV.
   3.1.26.7 Ribonuclease β₄.
   3.1.26.8 Ribonuclease M5.
   3.1.26.9 Ribonuclease (poly-(U)-specific).
   3.1.26.10 Ribonuclease IX.
   3.1.26.11 Ribonuclease Z.
3.1.27.—Endoribonucleases producing other than 5′-phosphomonoesters.
   3.1.27.1 Ribonuclease T(2).
   3.1.27.2 Bacillus subtilis ribonuclease.
   3.1.27.3 Ribonuclease T(1).
   3.1.27.4 Ribonuclease U(2).
   3.1.27.5 Pancreatic ribonuclease.
   3.1.27.6 Enterobacter ribonuclease.
   3.1.27.7 Ribonuclease F.
   3.1.27.8 Ribonuclease V.
   3.1.27.9 tRNA-intron endonuclease.
   3.1.27.10 rRNA endonuclease.
3.1.30.—Endoribonucleases active with either ribo- or deoxyribonucleic producing 5′ phosphomonoesters
   3.1.30.1 Aspergillus nuclease S(1).
   3.1.30.2 Serratia marcescens nuclease.
3.1.31.—Endoribonucleases active with either ribo- or deoxyribonucleic producing 3′ phosphomonoesters
   3.1.31.1 Micrococcal nuclease.

The enzyme is most preferably an exonuclease, such as a deoxyribonuclease, which cleave nucleic acids to form individual nucleotides. The advantages of exodeoxyribonucleases are that they are active on both single stranded and double stranded DNA and hydrolyse bases either in either the 5′-3′ or 3′-5′ direction.

An individual nucleotide is a single nucleotide. An individual nucleotide is one which is not bound to another nucleotide or nucleic acid by a nucleotide bond. A nucleotide bond involves one of the phosphate groups of a nucleotide being bound to the sugar group of another nucleotide. An individual nucleotide is typically one which is not bound by a nucleotide bond to another nucleic acid sequence of at least 5, at least 10, at least 20, at least 50, at least 100, at least 200, at least 500, at least 1000 or at least 5000 nucleotides.

Preferred enzymes for use in the method include exonuclease III enzyme from E. coli (SEQ ID NO: 10), exonuclease I from E. coli (SEQ ID NO: 12). RecJ from T. thermophilus (SEQ ID NO: 14) and bacteriophage lambda exonuclease (SEQ ID NO: 16) and variants thereof. The exonuclease enzyme preferably comprises any of the sequences shown in SEQ ID NOs: 10, 12, 14 and 16 or a variant thereof. Three identical subunits of SEQ ID NO: 16 interact to form a trimer exonuclease. A variant of SEQ ID NO: 10, 12, 14 or 16 is an enzyme that has an amino acid sequence which varies from that of SEQ ID NO: 10, 12, 14 or 16 and which retains nucleic acid handling ability. The enzyme may include modifications that facilitate handling of the nucleic acid and/or facilitate its activity at high salt concentrations and/or room temperature. The enzyme may include modifications that facilitate covalent attachment to or its interaction with the subunit. As discussed above, accessible cysteines may be removed from the enzyme to avoid non-specific reactions with a linker. Alternatively, one or more reactive cysteines may be introduced into the enzyme, for instance as part of a genetically-fused peptide linker, to facilitate attachment to the subunit.

Variants may differ from SEQ ID NO: 10, 12, 14 and 16 to the same extent as variants of SEQ ID NO: 2 differ from SEQ ID NO: 2 as discussed above.

A variant of SEQ ID NO: 10, 12, 14 or 16 retains its nucleic acid handling activity. A variant typically contains the regions of SEQ ID NO: 10, 12, 14 or 16 that are responsible for nucleic acid handling activity. The catalytic domains of SEQ ID NOs: 10, 12, 14 and 16 are discussed above. A variant of SEQ ID NO: 10, 12, 14 or 16 preferably comprises the relevant catalytic domain. A variant SEQ ID NO: 10, 12, 14 or 16 typically includes one or more modifications, such as substitutions, additions or deletions, outside the relevant catalytic domain.

Preferred enzymes that are capable of pushing or pulling the target nucleic acid sequence through the pore include polymerases, exonucleases, helicases and topoisomerases, such as gyrases. The polymerase is preferably a member of any of the Enzyme Classification (EC) groups 2.7.7.6, 2.7.7.7, 2.7.7.19, 2.7.7.48 and 2.7.7.49. The polymerase is preferably a DNA-dependent DNA polymerase, an RNA-dependent DNA polymerase, a DNA-dependent RNA polymerase or an RNA-dependent RNA polymerase. The helicase is preferably a member of any of the Enzyme Classification (EC) groups 3.6.1.- and 2.7.7.-. The helicase is preferably an ATP-dependent DNA helicase (EC group 3.6.1.8), an ATP-dependent RNA helicase (EC group 3.6.1.8) or an ATP-independent RNA helicase. The topoisomerase is preferably a member of any of the Enzyme Classification (EC) groups 5.99.1.2 and 5.99.1.3.

The enzyme may be labelled with a revealing label. The revealing label may be any of those described above.

The enzyme may be isolated from an enzyme-producing organism, such as E. coli, T. thermophilus or bacteriophage, or made synthetically or by recombinant means. For example, the enzyme may be synthesized by in vitro translation and transcription as described above and below. The enzyme may be produced in large scale following purification as described above.

### Preferred Constructs

Preferred constructs of the invention comprise the sequence shown in any one of SEQ ID NOs: 18, 20, 22, 24, 26, 28 and 30 or a variant thereof. Variants of SEQ ID NO: 18, 20, 22, 24, 26, 28 or 30 must retain their pore forming ability and nucleic acid handling ability. Variants may differ from SEQ ID NOs: 18, 20, 22, 24, 26, 28 and 30 to the same extent and in the same way as discussed above for variants of SEQ ID NO: 2 and variants of SEQ ID NO: 10, 12, 14 or 16.

### Polynucleotide Sequences

The present invention also provides polynucleotide sequences which encode a construct in which the enzyme is genetically fused to the subunit or is inserted into the sequence of the subunit. It is straightforward to generate such polynucleotide sequences using standard techniques. A polynucleotide sequence encoding the enzyme is either fused to or inserted into a polynucleotide sequence encoding the subunit. The fusion or insertion is typically in frame. If a polynucleotide sequence encoding the enzyme is inserted into a polynucleotide sequence encoding the subunit, the sequence encoding the enzyme is typically flanked at both ends by restriction endonuclease sites, such as those recognized by BspEI. It may also be flanked at both ends by polynucleotide sequences encoding linkers, such as 5 to 10 codons each encoding serine or glycine.

The polynucleotide sequence preferably encodes a construct comprising SEQ ID NO: 10, 12, 14 or 16 or a variant thereof genetically fused to or inserted into SEQ ID NO: 2 or a variant thereof. The variants of SEQ ID NO: 2, 10, 12, 14 or 16 may be any of those discussed above. SEQ ID NO: 10, 12, 14 or 16 or a variant thereof may be genetically fused to or inserted into SEQ ID NO: 2 or a variant thereof as described above.

The polynucleotide sequence preferably comprises SEQ ID NO: 9, 11, 13 or 15 or a variant thereof genetically fused to or inserted into SEQ ID NO: 1 or a variant thereof. SEQ ID NO: 9, 11, 13 or 15 or a variant thereof is preferably inserted into SEQ ID NO: 1 or a variant thereof between nucleotides 2765 and 2766, 2843 and 2844 or 2861 and 2862 of SEQ ID NO: 1. The polynucleotide sequence more preferably comprises the sequence shown in SEQ ID NO: 17, 19, 21, 23, 25, 27 or 29 or a variant thereof.

Variants of SEQ ID NOs: 1, 9, 11, 13, 15, 17, 19, 21, 23, 25, 27 or 29 are sequences that are at least 50%, 60%, 70%, 80%, 90% or 95% homologous based on nucleotide identity to sequence of SEQ ID NO: 1, 9, 11, 13, 15, 17, 19, 21, 23, 25, 27 or 29 over the entire sequence. There may be at least 80%, for example at least 85%, 90% or 95% nucleotide identity over a stretch of 600 or more, for example 700, 750, 850 or 900 or more, contiguous nucleotides (“hard homology”). Homology may be calculated as described above. The polynucleotide sequence may comprise a sequence that differs from SEQ ID NO: 1, 9, 11, 13, 15, 17, 19, 21, 23, 25, 27 or 29 on the basis of the degeneracy of the genetic code.

Polynucleotide sequences may be isolated and replicated using standard methods in the art. Chromosomal DNA may be extracted from a pore producing organism, such as Staphylococcus aureus, and/or an enzyme producing organism, such as E. coli, T. thermophilus or bacteriophage. The gene encoding the subunit and enzyme may be amplified using PCR involving specific primers. The amplified sequences may then be incorporated into a recombinant replicable vector such as a cloning vector. The vector may be used to replicate the polynucleotide in a compatible host cell. Thus polynucleotide sequences encoding a subunit and/or enzyme may be made by introducing a polynucleotide encoding a subunit and/or enzyme into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The polynucleotide sequence may be cloned into suitable expression vector. In an expression vector, the polynucleotide sequence encoding a construct is typically operably linked to a control sequence which is capable of providing for the expression of the coding sequence by the host cell. Such expression vectors can be used to express a construct.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The expression vector may then be introduced into a suitable host cell. Thus, a construct can be produced by inserting a polynucleotide sequence encoding a construct into an expression vector, introducing the vector into a compatible bacterial host cell, and growing the host cell under conditions which bring about expression of the polynucleotide sequence. The recombinantly-expressed construct may self-assemble into a pore in the host cell membrane. Alternatively, the recombinant construct produced in this manner may be isolated from the host cell and inserted into another membrane. When producing an oligomeric pore comprising a construct of the invention and at least one different subunit, the construct and different subunits may be expressed separately in different host cells as described above, removed from the host cells and assembled into a pore in a separate membrane, such as a rabbit cell membrane.

The vectors may be for example, plasmid, virus or phage vectors provided with an origin of replication, optionally a promoter for the expression of the said polynucleotide sequence and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The host cell typically expresses the construct at a high level. Host cells transformed with a polynucleotide sequence encoding a construct will be chosen to be compatible with the expression vector used to transform the cell. The host cell is typically bacterial and preferably E. coli. Any cell with a λ DE3 lysogen, for example C41 (DE3), BL21 (DE3), JM109 (DE3), B834 (DE3), TUNER, Origami and Origami B, can express a vector comprising the 17 promoter.

### Modified Pores

The present invention also provides modified pores for use in sequencing nucleic acids. The pores comprise at least one construct of the invention. The pores may comprise more than one, such as 2, 3 or 4, constructs of the invention.

A pore of the invention may be isolated, substantially isolated, purified or substantially purified. A pore of the invention is isolated or purified if it is completely free of any other components, such as lipids or other pores. A pore is substantially isolated if it is mixed with carriers or diluents which will not interfere with its intended use. For instance, a pore is substantially isolated or substantially purified if it present in a form that comprises less than 10%, less than 5%, less than 2% or less than 1% of other components, such as lipids or other pores. Alternatively, a pore of the invention may be present in a lipid bilayer or in a surfactant micelle.

The enzyme attached to the construct handles a target nucleic acid sequence in such a way that a proportion of the nucleotide in the target sequence interacts with the pore, preferably the barrel or channel of the pore. Nucleotides are then distinguished on the basis of the different ways in which they affect the current flowing through the pore during the interaction.

The fixed nature of the enzyme means that a target nucleic acid sequence is handled by the pore in a specific manner. For instance, each nucleotide may be digested from one of the target sequence in a processive manner or the target sequence may be pushed or pulled through the pore. This ensures that a proportion of the nucleotides in the target nucleic acid sequence interacts with the pore and is identified. The lack of any interruption in the signal is important when sequencing nucleic acids. In addition, the fixed nature of the enzyme and the pore means they can be stored together, thereby allowing the production of a ready-to-use sensor.

In a preferred embodiment, an exonuclease enzyme, such as a deoxyribonuclease, is attached to the pore such that a proportion of the nucleotides is released from the target nucleic acid and interacts with the barrel or channel of the pore. In another preferred embodiment, an enzyme that is capable of pushing or pulling the target nucleic acid sequence through the pore is attached to the pore such that the target nucleic acid sequence is pushed or pulled through the barrel or channel of the pore and a proportion of the nucleotides in the target sequence interacts with the barrel or channel. In this embodiment, the nucleotides may interact with the pore in blocks or groups of more than one, such as 2, 3 or 4. Suitable enzymes include, but are not limited to, polymerases, exonucleases, helicases and topoisomerases, such as gyrases. In each embodiment, the enzyme is preferably attached to the pore at a site in close proximity to the opening of the barrel of channel of the pore. The enzyme is more preferably attached to the pore such that its active site is orientated towards the opening of the barrel of channel of the pore. This means that a proportion of the nucleotides of the target nucleic acid sequence is fed in the barrel or channel. The enzyme is preferably attached to the cis side of the pore.

The modified pore may be based on any of the transmembrane protein pores discussed above, including the β-barrel pores and α-helix bundle pores.

For constructs comprising the sequence shown in SEQ ID NO: 2 or a variant thereof, the pore typically comprises an appropriate number of additional subunits comprising the sequence shown in SEQ ID NO: 2 or a variant thereof. A preferred pore of the invention comprises one construct comprising the sequence shown in SEQ ID NO: 2 or a variant thereof and six subunits comprising the sequence shown in SEQ ID NO: 2 or a variant thereof. The pore may comprise one or more subunits comprising the sequence shown in SEQ ID NO: 4 or a variant thereof. SEQ ID NO: 4 shows the sequence of SEQ ID NO: 2 except that it has an arginine at position 113 (M113R) and a glutamine at position 139 (N139Q). A variant of SEQ ID NO: 4 may differ from SEQ ID NO: 4 in the same way and to the same extent as discussed for SEQ ID NO: 2 above. A preferred pore of the invention comprises one construct comprising the sequence shown in SEQ ID NO: 2 or a variant thereof and six subunits comprising the sequence shown in SEQ ID NO: 4 or a variant thereof.

The pores may comprise a molecular adaptor that facilitates the interaction between the pore and the nucleotides or the target nucleic acid sequence. The presence of the adaptor improves the host-guest chemistry of the pore and nucleotides released from or present in the target nucleic acid sequence. The principles of host-guest chemistry are well-known in the art. The adaptor has an effect on the physical or chemical properties of the pore that improves its interaction with nucleotides. The adaptor typically alters the charge of the barrel or channel of the pore or specifically interacts with or binds to nucleotides thereby facilitating their interaction with the pore.

The adaptor mediates the interaction between nucleotides released from or present in the target nucleic acid sequence and the pore. The nucleotides preferably reversibly bind to the pore via or in conjunction with the adaptor. The nucleotides most preferably reversibly bind to the pore via or in conjunction with the adaptor as they pass through the pore across the membrane. The nucleotides can also reversibly bind to the barrel or channel of the pore via or in conjunction with the adaptor as they pass through the pore across the membrane. The adaptor preferably constricts the barrel or channel so that it may interact with the nucleotides.

The adaptor is typically cyclic. The adaptor preferably has the same symmetry as the pore. An adaptor having seven-fold symmetry is typically used if the pore is heptameric (e.g. has seven subunits around a central axis that contribute 14 strands to a transmembrane β barrel). Likewise, an adaptor having six-fold symmetry is typically used if the pore is hexameric (e.g. has six subunits around a central axis that contribute 12 strands to a transmembrane β barrel, or is a 12-stranded β barrel). Any adaptor that that facilitates the interaction between the pore and the nucleotide can be used. Suitable adaptors include, but are not limited to, cyclodextrins, cyclic peptides and cucurbiturils. The adaptor is preferably a cyclodextrin or a derivative thereof. The adaptor is more preferably heptakis-6-amino-β-cyclodextrin (am₇-βCD), 6-monodeoxy-6-monoamino-β-cyclodextrin (am₁-βCD) or heptakis-(6-deoxy-6-guanidino)-cyclodextrin (gu₇-βCD). Table 2 below shows preferred combinations of pores and adaptors.

TABLE 2
Suitable combinations of pores and adaptors

| | Number of | |
|---|---|---|
| | strands in the | |
| | transmembrane | |
| Pore | β-barrel | Adaptor |
| Leukocidin | 16 | γ-cyclodextrin (γ-CD) |
| OmpF | 16 | γ-cyclodextrin (γ-CD) |
| α-hemolysin | 14 | β-cyclodextrin (β-CD) |
| (or a variant | | 6-monodeoxy-6- |
| thereof | | monoamino-β-cyclodextrin |
| discussed | | (am₁β-CD) |
| above) | | heptakis-6-amino-β- |
| | | cyclodextrin (am₇-β-CD) |
| | | heptakis-(6-deoxy-6- |
| | | guanidino)-cyclodextrin |
| | | (gu₇-β-CD) |
| OmpG | 14 | β-cyclodextrin (β-CD) |
| | | 6-monodeoxy-6- |
| | | monoamino-β-cyclodextrin |
| | | (am₁β-CD) |
| | | heptakis-6-amino-β- |
| | | cyclodextrin (am₇-β-CD) |
| | | heptakis-(6-deoxy-6- |
| | | guanidino)-cyclodextrin |
| | | (gu₇-β-CD) |
| NalP | 12 | α-cyclodextrin (α-CD) |
| OMPLA | 12 | α-cyclodextrin (α-CD) |

The adaptor is preferably covalently attached to the pore. The adaptor can be covalently attached to the pore using any method known in the art. The adaptor may be attached directly to the pore. The adaptor is preferably attached to the pore using a bifunctional crosslinker. Suitable crosslinkers are well-known in the art. Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate. The most preferred crosslinker is succinimidyl 3-(2-pyridyldithio)propionate (SPDP). Typically, the adaptor is covalently attached to the bifunctional crosslinker before the adaptor/crosslinker complex is covalently attached to the pore but it is also possible to covalently attach the bifunctional crosslinker to the pore before the bifunctional crosslinker/pore complex is attached to the adaptor.

The site of covalent attachment is selected such that the adaptor facilitates interaction of nucleotides released from or present in the target nucleic acid sequence with the pore and thereby allows detection of nucleotides. This can be done as explained in the co-pending International application claiming priority from U.S. Application No. 61/078,687 and being filed simultaneously with this application [J A Kemp & Co Ref: N.104403A; Oxford Nanolabs Ref: ONL IP 004].

For pores based on α-HL, the correct orientation of the adaptor within the barrel or channel of the pore and the covalent attachment of adaptor to the pore can be facilitated as described in the co-pending International application claiming priority from U.S. Application No. 61/078,687 and being filed simultaneously with this application [J A Kemp & Co Ref: N.104403A; Oxford Nanolabs Ref: ONL IP 004]. Any of the specific modifications to SEQ ID NO: 2 disclosed in the co-pending application are equally applicable to the pores of this invention. In particular, every subunit of the pore, including the construct(s), preferably has a glutamine at position 139 of SEQ ID NO: 2. One or more of the subunits of the pore, including the construct(s), may have an arginine at position 113 of SEQ ID NO: 2. One or more of the subunits of the pore, including the construct(s), may have a cysteine at position 119, 121 or 135 of SEQ ID NO: 2. Any of the variants of SEQ ID NO: 2 shown in SEQ ID NOs: 4, 6, 8, 10, 12 and 14 of the co-pending application may be used to form a modified pore of the invention.

Preferred modified pores of the invention comprise:
(a) a construct comprising the sequence shown in SEQ ID NO: 18, 20, 22, 24, 26, 28 or 30 or a variant thereof and six subunits of α-HL M113R/N139Q shown in SEQ ID NO: 4;
(b) a construct of the invention comprising the sequence shown in SEQ ID NO: 2 or a variant thereof, five subunits of α-HL M113R/N139Q shown in SEQ ID NO: 4 or a variant thereof and one subunit of α-HL M113R/N139Q/G119C-D8 shown in SEQ ID NO: 10 of the co-pending application;
(c) a a construct of the invention comprising the sequence shown in SEQ ID NO: 2 or a variant thereof, five subunits of α-HL M113R/N139Q shown in SEQ ID NO: 4 or a variant thereof and one subunit of α-HL M113R/N139Q/N121C-D8 shown in SEQ ID NO: 12 of the co-pending application; or
(d) a construct of the invention comprising the sequence shown in SEQ ID NO: 2 or a variant thereof, five subunits of α-HL M113R/N139Q shown in SEQ ID NO: 4 or a variant thereof and and one subunit of α-HL M113R/N139Q/L135C-D8 shown in SEQ ID NO: 14 of the co-pending application.

### Methods of Producing Constructs of the Invention

The invention also provides methods of producing a construct of the invention. The methods comprise covalently attaching a nucleic acid handling enzyme to a transmembrane protein pore subunit. Any of the subunits and enzymes discussed above can be used in the methods. The site of and method of covalent attachment are selected as discussed above.

The methods also comprise determining whether or not the construct is capable of forming a pore and handling nucleic acids. Assays for doing this are described above. If a pore can be formed and nucleic acids can be handled, the subunit and enzyme have been attached correctly and a construct of the invention has been produced. If a pore cannot be formed or nucleic acids cannot be handled, a construct of the invention has not been produced.

### Methods of Producing Modified Pores

The present invention also provides methods of producing modified pores of the invention. The modified pore may be formed by allowing at least one construct of the invention to form a pore with other suitable subunits or by covalently attaching an enzyme to a subunit in an oligomeric pore. Any of the constructs, subunits, enzymes or pores discussed above can be used in the methods. The site of and method of covalent attachment are selected as discussed above.

The methods also comprise determining whether or not the pore is capable of handling nucleic acids and detecting nucleotides. The pore may be assessed for its ability to detect individual nucleotides or short chains of nucleotides, such as di- or trinucleotides. Assays for doing this are described above and below. If the pore is capable of handling nucleic acids and detecting nucleotides, the subunit and enzyme have been attached correctly and a pore of the invention has been produced. If a pore cannot be handle nucleic acids and detect nucleotides, a pore of the invention has not been produced.

In a preferred embodiment, a heteroheptamer of seven subunits comprising the sequence shown in SEQ ID NO: 2 or a variant thereof and containing one cysteine in an appropriate place is reacted with a bifunctional cross-linker. The pore may be reacted with the linker before or after it has been purified, typically by SDS PAGE. The pore/linker construct is then reacted with an enzyme containing at least one reactive cysteine, for instance on a genetically-fused peptide linker. After the coupling reaction, the modified pore of the invention is removed from any unreacted enzyme or pore/linker construct.

### Method of Purifying Pores

The present invention also provides methods of purifying modified pores of the invention. The methods allow the purification of pores comprising at least one construct of the invention. The methods do not involve the use of anionic surfactants, such as sodium dodecyl sulphate (SDS), and therefore avoid any detrimental effects on the enzyme part of the construct. The methods are particularly good for purifying pores comprising a construct of the invention in which the subunit and enzyme have been genetically fused.

The methods involve providing at least one construct of the invention and any remaining subunits required to form a pore of the invention. Any of the constructs and subunits discussed above can be used. The construct(s) and remaining subunits are inserted into synthetic lipid vesicles and allowed to oligomerise. Methods for inserting the construct(s) and remaining subunits into synthetic vesicles are well known in the art.

The synthetic vesicles should have similar properties to rabbit cell membranes, but should lack the rabbit cell membrane proteins. The vesicles may comprise any components and are typically made of a blend of lipids. Suitable lipids are well-known in the art. The synthetic vesicles preferably comprise 30% cholesterol, 30% phosphatidylcholine (PC), 20% phosphatidylethanolamine (PE), 10% sphingomyelin (SM) and 10% phosphatidylserine (PS).

The vesicles are then contacting with a non-ionic surfactant or a blend of non-ionic surfactants. The non-ionic surfactant is preferably an Octyl Glucoside (OG) or DoDecyl Maltoside (DDM) detergent. The oligomerised pores are then purified, for example by using affinity purification based on his-tag or Ni-NTA.

### Methods of Sequencing Nucleic Acids

The present invention also provides methods of sequencing a target nucleic acid sequence. In one embodiment, the method comprises (a) contacting the target sequence with a pore of the invention, which comprises an exonuclease, such that the exonuclease digests an individual nucleotide from one end of the target sequence; (b) contacting the nucleotide with the pore so that the nucleotide interacts with the adaptor, (c) measuring the current passing through the pore during the interaction and thereby determining the identity of the nucleotide; and (d) repeating steps (a) to (c) at the same end of the target sequence and thereby determining the sequence of the target sequence. Hence, the method involves stochastic sensing of a proportion of the nucleotides in a target nucleic acid sequence in a successive manner in order to sequence the target sequence. Individual nucleotides are described above.

In another embodiment, the method comprises (a) contacting the target sequence with a pore of the invention so that the target sequence is pushed or pulled through the pore and a proportion of the nucleotides in the target sequence interacts with the pore and (b) measuring the current passing through the pore during each interaction and thereby determining the sequence of the target sequence. Hence, the method involves stochastic sensing of a proportion of the nucleotides in a target nucleic acid sequence as the nucleotides pass through the barrel or channel in a successive manner in order to sequence the target sequence.

Pores comprising a construct of the invention are particularly suited to these methods. In order to effectively sequence the nucleic acid, it is important to ensure that a proportion of the nucleotides in the nucleic acid is identified in a successive manner. The fixed nature of the enzyme means that a proportion of the nucleotides in the target sequence affects the current flowing through the pore.

The whole or only part of the target nucleic acid sequence may be sequenced using this method. The nucleic acid sequence can be any length. For example, the nucleic acid sequence can be at least 10, at least 50, at least 100, at least 150, at least 200, at least 250, at least 300, at least 400 or at least 500 nucleotides in length. The nucleic acid sequence can be naturally occurring or artificial. For instance, the method may be used to verify the sequence of a manufactured oligonucleotide. The methods are typically carried out in vitro.

The methods may be carried out using any suitable membrane/pore system in which a pore comprising a construct of the invention is inserted into a membrane. The methods are typically carried out using (i) an artificial membrane comprising a pore comprising a construct of the invention, (ii) an isolated, naturally occurring membrane comprising a pore comprising a construct of the invention, or (iii) a cell expressing a pore comprising a construct of the invention. The methods are preferably carried out using an artificial membrane. The membrane may comprise other transmembrane and/or intramembrane proteins as well as other molecules in addition to the pore of the invention.

The membrane forms a barrier to the flow of ions, nucleotides and nucleic acids. The membrane is preferably a lipid bilayer. Lipid bilayers suitable for use in accordance with the invention can be made using methods known in the art. For example, lipid bilayer membranes can be formed using the method of Montal and Mueller (1972). Lipid bilayers can also be formed using the method described in International Application No. PCT/GB08/000563.

The methods of the invention may be carried out using lipid bilayers formed from any membrane lipid including, but not limited to, phospholipids, glycolipids, cholesterol and mixtures thereof. Any of the lipids described in International Application No. PCT/GB08/000563 may be used.

Methods are known in the art for inserting pores into membranes, such as lipid bilayers. Some of those methods are discussed above.

### Interaction Between the Pore and Nucleotides

The nucleotide or nucleic acid may be contacted with the pore on either side of the membrane. The nucleotide or nucleic acid may be introduced to the pore on either side of the membrane. The nucleotide or nucleic acid is typically contacted with the side of the membrane on which the enzyme is attached to the pore. This allows the enzyme to handle the nucleic acid during the method.

A proportion of the nucleotides of the target nucleic acid sequence interacts with the pore and/or adaptor as it passes across the membrane through the barrel or channel of the pore. Alternatively, if the target sequence is digested by an exonuclease, the nucleotide may interact with the pore via or in conjunction with the adaptor, dissociate from the pore and remain on the same side of the membrane. The methods may involve the use of pores in which the orientation of the adaptor is fixed. In such embodiments, the nucleotide is preferably contacted with the end of the pore towards which the adaptor is oriented. Most preferably, the nucleotide is contacted with the end of the pore towards which the portion of the adaptor that interacts with the nucleotide is orientated.

The nucleotides may interact with the pore in any manner and at any site. As discussed above, the nucleotides preferably reversibly bind to the pore via or in conjunction with the adaptor. The nucleotides most preferably reversibly bind to the pore via or in conjunction with the adaptor as they pass through the pore across the membrane. The nucleotides can also reversibly bind to the barrel or channel of the pore via or in conjunction with the adaptor as they pass through the pore across the membrane.

During the interaction between a nucleotides and the pore, the nucleotide affects the current flowing through the pore in a manner specific for that nucleotide. For example, a particular nucleotide will reduce the current flowing through the pore for a particular mean time period and to a particular extent. In other words, the current flowing through the pore is distinctive for a particular nucleotide. Control experiments may be carried out to determine the effect a particular nucleotide has on the current flowing through the pore. Results from carrying out the method of the invention on a test sample can then be compared with those derived from such a control experiment in order to identify a particular nucleotide.

### Apparatus

The methods may be carried out using any apparatus that is suitable for investigating a membrane/pore system in which a pore comprising a construct of the invention is inserted into a membrane. The methods may be carried out using any apparatus that is suitable for stochastic sensing. For example, the apparatus comprises a chamber comprising an aqueous solution and a barrier that separates the chamber into two sections. The barrier has an aperture in which the membrane containing the pore is formed. The nucleotide or nucleic acid may be contacted with the pore by introducing the nucleic acid into the chamber. The nucleic acid may be introduced into either of the two sections of the chamber, but is preferably introduced into the section of the chamber containing the enzyme.

The methods may be carried out using the apparatus described in International Application No. PCT/GB08/000562.

The methods involve measuring the current passing through the pore during interaction with the nucleotides. Therefore the apparatus also comprises an electrical circuit capable of applying a potential and measuring an electrical signal across the membrane and pore. The methods may be carried out using a patch clamp or a voltage clamp. The methods preferably involves the use of a voltage clamp.

### Conditions

The methods of the invention involve the measuring of a current passing through the pore during interaction with nucleotides in a target nucleic acid sequence. Suitable conditions for measuring ionic currents through transmembrane protein pores are known in the art and disclosed in the Examples. The method is carried out with a voltage applied across the membrane and pore. The voltage used is typically from −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 120 mV to 170 mV. It is possible to increase discrimination between different nucleotides by a pore of the invention by using an increased applied potential.

The methods are carried out in the presence of any alkali metal chloride salt. In the exemplary apparatus discussed above, the salt is present in the aqueous solution in the chamber. Potassium chloride (KCl), sodium chloride (NaCl) or caesium chloride (CsCl) is typically used. KCl is preferred. The salt concentration is typically from 0.1 to 2.5M, from 0.3 to 1.9M, from 0.5 to 1.8M, from 0.7 to 1.7M, from 0.9 to 1.6M or from 1 M to 1.4M. High salt concentrations provide a high signal to noise ratio and allow for currents indicative of the presence of a nucleotide to be identified against the background of normal current fluctuations. However, lower salt concentrations are preferably used so that the enzyme is capable of functioning. The salt concentration is preferably from 150 to 500 mM. Good nucleotide discrimination at these low salt concentrations can be achieved by carrying out the method at temperatures above room temperature, such as from 30° C. to 40° C.

The methods are typically carried out in the presence of a buffer. In the exemplary apparatus discussed above, the buffer is present in the aqueous solution in the chamber. Any buffer may be used in the methods. One suitable buffer is Tris-HCl buffer. The methods are typically carried out at a pH of from 4.0 to 10.0, from 4.5 to 9.5, from 5.0 to 9.0, from 5.5 to 8.8, from 6.0 to 8.7 or from 7.0 to 8.8 or 7.5 to 8.5. The pH-used is preferably about 7.5.

The methods are typically carried out at from 0° C. to 100° C., from 15° C. to 95° C., from 16° C. to 90° C. from 17° C. to 85° C., from 18° C. to 80° C., 19° C. to 70° C., or from 20° C. to 60° C. The methods may be carried out at room temperature. The methods are preferably carried out at a temperature that supports enzyme function, such as about 37° C. Good nucleotide discrimination can be achieved at low salt concentrations if the temperature is increased.

In addition to increasing the solution temperature, there are a number of other strategies that can be employed to increase the conductance of the solution, while maintaining conditions that are suitable for enzyme activity. One such strategy is to use the lipid bilayer to divide two different concentrations of salt solution, a low salt concentration of salt on the enzyme side and a higher concentration on the opposite side. One example of this approach is to use 200 mM of KCl on the cis side of the membrane and 500 mM KCl in the trans chamber. At these conditions, the conductance through the pore is expected to be roughly equivalent to 400 mM KCl under normal conditions, and the enzyme only experiences 200 mM if placed on the cis side. Another possible benefit of using asymmetric salt conditions is the osmotic gradient induced across the pore. This net flow of water could be used to pull nucleotides into the pore for detection. A similar effect can be achieved using a neutral osmolyte, such as sucrose, glycerol or PEG. Another possibility is to use a solution with relatively low levels of KCl and rely on an additional charge carrying species that is less disruptive to enzyme activity.

### Exonuclease-Based Methods

In one embodiment, the method of sequencing a target nucleic acid sequence involves contacting the target sequence with a pore having an exonuclease enzyme, such as deoxyribonuclease, attached thereto. The constructs needed to make such pores are discussed above. Any of the exonuclease enzymes discussed above may be used in the method. The exonuclease releases individual nucleotides from one end of the target sequence. Exonucleases are enzymes that typically latch onto one end of a nucleic acid sequence and digest the sequence one nucleotide at a time from that end. The exonuclease can digest the nucleic acid in the 5′ to 3′ direction or 3′ to 5′ direction. The end of the nucleic acid to which the exonuclease binds is typically determined through the choice of enzyme used and/or using methods known in the art. Hydroxyl groups or cap structures at either end of the nucleic acid sequence may typically be used to prevent or facilitate the binding of the exonuclease to a particular end of the nucleic acid sequence.

The method involves contacting the nucleic acid sequence with the exonuclease so that the nucleotides are digested from the end of the nucleic acid at a rate that allows identification of a proportion of nucleotides as discussed above. Methods for doing this are well known in the art. For example, Edman degradation is used to successively digest single amino acids from the end of polypeptide such that they may be identified using High Performance Liquid Chromatography (HPLC). A homologous method may be used in the present invention.

The rate at which the exonuclease functions is typically slower than the optimal rate of a wild-type exonuclease. A suitable rate of activity of the exonuclease in the method of sequencing involves digestion of from 0.5 to 1000 nucleotides per second, from 0.6 to 500 nucleotides per second, 0.7 to 200 nucleotides per second, from 0.8 to 100 nucleotides per second, from 0.9 to 50 nucleotides per second or 1 to 20 or 10 nucleotides per second. The rate is preferably 1, 10, 100, 500 or 1000 nucleotides per second. A suitable rate of exonuclease activity can be achieved in various ways. For example, variant exonucleases with a reduced optimal rate of activity may be used in accordance with the invention.

### Pushing or Pulling DNA Through the Pore

Strand sequencing involves the controlled and stepwise translocation of nucleic acid polymers through a pore. The majority of DNA handling enzymes are suitable for use in this application provided they hydrolyse, polymerize or process single stranded DNA or RNA. Preferred enzymes are polymerases, exonucleases, helicases and topoisomerases, such as gyrases. The enzyme moiety is not required to be in as close a proximity to the pore lumen as for individual nucleotide sequencing as there is no potential for disorder in the series in which nucleotides reach the sensing moiety of the pore.

The two strategies for single strand DNA sequencing are the translocation of the DNA through the nanopore, both cis to trans and trans to cis, either with or against an applied potential. The most advantageous mechanism for strand sequencing is the controlled translocation of single strand DNA through the nanopore with an applied potential. Exonucleases that act progressively or processively on double stranded DNA can be used on the cis side of the pore to feed the remaining single strand through under an applied potential or the trans side under a reverse potential. Likewise, a helicase that unwinds the double stranded DNA can also be used in a similar manner. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential.

### Kits

The present invention also provides kits for producing a modified pore for use in sequencing nucleic acids. In one embodiment, the kits comprise at least one construct of the invention and any remaining subunits need to form a pore. The kits may comprise enough constructs of the invention to form a complete pore (i.e. a homo-oligomer). The kits may comprise any of the constructs and subunits discussed above. A preferred kit comprises (i) a construct comprising a subunit comprising the sequence shown in SEQ ID NO: 2 or a variant thereof and (ii) six subunits comprising the sequence shown in SEQ ID NO: 2 or a variant thereof. A more preferred kit comprises (i) a construct comprising the sequence shown in SEQ ID NO: 18, 20, 22, 24, 26, 28 or 30 or a variant thereof and (ii) six subunits comprising the sequence shown in SEQ ID NO: 2 or a variant thereof.

In another embodiment, the kits comprise at least one polynucleotide sequence of the invention and polynucleotide sequences encoding any remaining subunits needed to form a pore. The kit may comprise enough polynucleotides of the invention to encode a complete pore (i.e. a homo-oligomer). The kits may comprise any of the polynucleotides described above. A preferred kit comprises (i) a polynucleotide sequence encoding a construct, which comprises a subunit comprising the sequence shown in SEQ ID NO: 2 or a variant thereof and (ii) six polynucleotide sequences each encoding a subunit comprising the sequence shown in SEQ ID NO: 2 or a variant thereof. A more preferred kit comprises (i) a polynucleotide sequence encoding a construct comprising the sequence shown in SEQ ID NO: 18, 20, 22, 24, 26, 28 or 30 or a variant thereof and (ii) six polynucleotide sequences each encoding a subunit comprising the sequence shown in SEQ ID NO: 2 or a variant thereof.

The kits of the invention may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotide sequences, a membrane as defined above or voltage or patch clamp apparatus. Reagents may be present in the kit in a dry state such that a fluid sample resuspends the reagents. The kit may also, optionally, comprise instructions to enable the kit to be used in the method of the invention or details regarding which patients the method may be used for. The kit may, optionally, comprise nucleotides.

The following Example illustrates the invention:

### Example

### 1 Materials and Methods

### 1.1 Bacterial Strains and Growth Conditions

The bacterial strains used in this work were E. coli strains XL-10 Gold and BL21 DE3 pLysS (Stratagene). E. coli strains were grown at 37° C. either in Luria-Bertani Broth (LB), Terrific Broth at 225 rpm, Luria-Bertani agar (LA) or tryptone-yeast extract agar (TY) (Bertani, G. (1951). Studies on lysogenesis. I. The mode of phage liberation by lysogenic Escherichia coli. Journal of Bacteriology. 62, 293-300; Beringer, J. (1974). R factor transfer in Rhizobium leguminosarum. Journal of General Microbiology. 84, 188-98; and Tartoff, K. and Hobbs, C. (1987). Improved media for growing plasmid and cosmid clones. Bethesda Research Labs Focus. 9, 12). Antibiotics were used at the following concentrations: Ampicillin 100 μg ml⁻¹; chloramphenicol 30 μg ml⁻¹.

### 1.2 Genetic Manipulations

All general DNA cloning was performed as adapted methods of that previously described (Sambrook, J. and Russell, D. (2001). Molecular Cloning: A Laboratory Manual, 3rd Edition. Cold Spring Harbor Laboratory Press, Cold Spring Harbor, NY). DNA polymerases, restriction endonucleases, exonuclease, ligases and phosphatases were all obtained from New England Biolabs. Exonuclease genes were manufactured by GenScript Corporation and received as fragments cloned into pT7-SC1, by BspEI or NdeI/HindIII. All mutations and fusion constructs were assembled in the expression vector pT7-SC1 (Cheley, S., Malghani. M., Song. L., Hobaugh, M., Gouaux, E., Yang, J. and Bayley, H. (1997). Spontaneous oligomerization of a staphylococcal alpha-hemolysin conformationally constrained by removal of residues that form the transmembrane beta-barrel. Protein Engineering. 10, 1433-43) and verified by sequencing using either the T7 forward or reverse primers, EcoExoIII_seq and EcoExoI_seq.

Site directed mutagenesis of the αHL gene was performed by in vivo homologous recombination of PCR products (Jones. D. (1995) PCR mutagenesis and recombination in vivo. In PCR primer: a laboratory manual. In: Dveksler, C. (ed). Cold Spring Harbor Laboratory Press, Cold Spring Harbor, NY). Amplification of two halves of the target plasmid with complimentary primer pairs generates two PCR products with complimentary sequences at both the 5′ and 3′ ends. Transformation of both products into chemically competent E. coli allows in vivo homologous recombination. For all mutagenesis SC46 was used as the antisense primer for amplification of product 1 and SC47 as the sense primer for amplification of product 2. These complementary primer binding sites are within the β-lactamase gene of pT7-SC1. Colonies recovered on LA 100 ng μl⁻¹ ampicillin therefore indicated successful homologous recombination.

PCR was conducted in 50 μl reactions using 1 unit Phusion™ DNA polymerase, 0.2 mM dNTPs, 1 μM primers and 4 ng BamHI/HindIII or NdeI/EcoNI digested plasmid DNA. Reactions were cycled as follows: 1 cycle of 98° C. for 2 min; 30 cycles of 98° C. for 15 s, 57° C. for 30 s and 72° C. for 45 s; and a final extension of 72° C. for 5 min. 2.5 μl of each pair of PCR products were mixed and used to transform chemically competent E. coli (XL-10 Gold).

### 1.3 Rapid In Vitro Transcription Translation

[³⁵S]L-methionine labelled proteins were generated by coupled in vitro transcription and translation (IVTT) using an E. coli T7-S30 extract system for circular DNA (Promega). The complete amino acid mixture (1 mM) minus cysteine and the complete amino acid mixture (1 mM) minus methionine, supplied in the kit, were mixed in equal volumes to obtain the working amino acid solution required to generate high concentrations of the protein. Reactions were scaled up or down based on the following, for a 50 μl reaction volume: 20 μl S30 Premix solution; 5 μl amino acid mix; 1 μl [³⁵S]L-methionine (MP Biomedicals, 1175 Ci mmol⁻¹, 10 mCi ml⁻¹), 1 μl rifampicin (0.8 mg ml⁻¹), 8 μl plasmid DNA (400 ng μl⁻¹) and 15 μl T7 S30 extract. Synthesis was carried out for 1.5 hours at 37° C. to produce 50 μl of radiolabelled IVTT protein. Different proteins were also co-expressed in one reaction as for coupled transcription, translation and oligomerisation. The reaction components remained the same except the DNA concentration was divided accordingly for each plasmid encoding each protein. Protein samples were centrifuged at 14,000 rpm for 10 minutes to separate insoluble debris of IVTT reactions.

### 1.4 In Vivo Protein Expression

Wild-type α-hemolysin and fusion constructs were cloned into the expression vector pT7-SC1, under the control of the inducible T7 promoter, and expressed in E. coli (BL21 DE3 pLysS, Stratagene) as soluble proteins. Cultures were grown to a high OD₆₀₀ (approximately 1.5-2) at 37° C. and 240 rpm in Terrific broth medium (100 μg μl⁻¹ ampicillin and 30 μg μl⁻¹ chloramphenicol). The temperature was reduced to 18° C. and cultures left for 30 minutes to equilibrate. Over expression of the target protein was induced by addition of IPTG to the medium (0.2 mM). After 18 hours cells were pelleted at 10,000 rpm for 30 minutes at 4° C. Cells were resuspended and lysed by the addition of BugBuster (Novagen) supplemented with the addition of benzonase, EDTA-free proteinase inhibitors (Roche) and to 50 mM MgCl₂. Cell debris was pelleted by centrifugation at 10.000 rpm for 30 minutes at 4° C. and polyethyleneimine (PET) added to the supernatant. The recovered supernatant was incubated for 30 mins at 4° C. after which precipitate was removed by centrifugation at 10,000 rpm for 30 minutes at 4° C. Clarified lysate was filtered and adjusted to pH 8.0, 500 mM NaCl, 10 mM Imidazole.

His-tagged proteins were purified as standard practice by Ni-NTA affinity chromatography and gel filtration. Non-tagged α-hemolysin subunits were purified as standard practice by cation exchange followed by gel filtration.

### 1.4.1 Affinity Purification (His-Tag

Clarified lysate was filtered and adjusted to pH 8.0, 500 mM NaCl, 10 mM Imidazole before loading onto a His-Trap crude column (GE Healthcare) and eluted with 300 mM Imidazole. Fractions containing the protein of interest were combined and applied to a gel filtration column equilibrated with 10 mM TRIS pH 8.0, 100 mM NaCl, 1 mM DTT. Eluted protein was evaluated by SDS-PAGE.

### 1.4.2 Ion Exchange

Clarified lysate was filtered and adjusted to 10 mM MES pH 6.0 before loading onto a cation exchange column (GE Healthcare) and eluting with 0-500 mM NaCl. Fractions containing the protein of interest were combined and applied to a gel filtration column. Eluted protein was evaluated by SDS-PAGE.

To maintain the reactivity of engineered cysteine residues in a-Hemolysin derivatives, required as sites for chemical modification, proteins were purified using the same buffers but supplemented to 1 mM DTT. Exonucleases or exonuclease fusion proteins were purified using the same buffers supplemented to 1 mM MgCl₂.

### 1.5 Oligomerisation on Red Blood Cell Membranes

α-Hemolysin monomers were mixed in various molar ratios and allowed to oligomerise on rabbit erythrocyte membranes (2.5 mg protein ml⁻¹) for 1 hour at either room temperature, 30° C., 37° C. or 42° C. After the incubation, reaction mixture was centrifuged at 14,000 rpm for 10 minutes and supernatant discarded. Membrane pellet was washed by resuspension in 200 μl MBSA (10 mM MOPS, 150 mM NaCl, pH 7.4 containing 1 mg ml⁻¹ bovine serum albumin) and centrifuging again at 14,000 rpm for 10 minutes. After discarding the supernatant, membrane pellet was dissolved in 75 μl of 1×Laemmli sample buffer, with the addition of β-mercaptoethanol. The entire sample was loaded into a single well of a 5% SDS-polyacrylamide gel and electrophoresed for ˜18 hours at 50 V, with 0.01 mM sodium thioglycolate included in the running buffer. Gel was vacuum-dried onto a Whatman 3 mm filter paper at 50° C. for about three hours and exposed to an X-ray film overnight (Kodak). The oligomer band was excised from the gel, using the autoradiogram as template, and the gel slice rehydrated in 300 μl TE buffer (10 mM Tris, 1 mM EDTA, pH 8.0) containing 2 mM DTT. After removing the Whatman filter paper slice, gel piece was crushed using a sterile pestle. Oligomer protein was separated from gel debris by centrifuging through 0.2 UM cellulose acetate microfilterage tubes (Rainin) at 14,000 rpm for 30 min. Filtrate was stored in aliquots at −80° C.

### 1.6 Oligomerisation on Synthetic Lipid Vesicles

Synthetic lipid vesicles composed of: 30% cholesterol; 30% phosphatidylcholine (PC); 20% phosphatidylethanolamine (PE); 10% sphingomyelin (SM); 10% phosphatidylserine (PS); were prepared by bath sonication for 15 minutes at room temperature. Organic solvent is evaporated by a gentle stream of nitrogen until a dry film is produced. Deionised water added to give a required concentration of 2.5 mg ml⁻¹ and mixture bath sonicated again for 15 minutes. Wild-type α-hemolysin and fusion monomers were mixed in various molar ratios and allowed to oligomerise on synthetic lipid vesicles (2.5 mg ml⁻¹ for every 1 mg α-hemolysin monomer) for 1 hour at either room temperature, 30° C., 37° C. or 42° C. and 350 rpm. To pellet lipid associated proteins samples were centrifuged at 14,000 rpm for 10 minutes. Pellet was washed once in MBSA (10 mM MOPS, 150 mM NaCl, pH 7.4 containing 1 mg ml⁻¹ bovine serum albumin) and lipids were dissolved by addition of 0.1-1% n-Dodecyl-D-maltopyranoside (DDM), for 1 hour at either 4° C. or room temperature. To purify the fusion homo and heteroheptamers away from wild-type homoheptamer 300 μl of Ni-NTA agarose (Qiagen) was added and left overnight at 4° C. and 350 rpm. Affinity bound heptamer was pelted with Ni-NTA agarose by centrifugation at 14,000 rpm for 10 minutes. The Ni-NTA agarose beads were washed twice in 500 μl wash buffer (10 mM Tris, 10 mM Imidazole, 500 mM NaCl, pH 8.0) for 10 minutes and recovered by centrifugation. Purified heteroheptamer was eluted in 500 μelution buffer (10 mM Tris, 250 mM Imidazole, pH 8.0) for 1 hour at 4° C., The Ni-NTA agarose was removed by centrifugation and the supernatant containing the eluted purified fusion heptamers removed. Eluted heptamers were de-salted by passage through a buffer exchange column (NAP-5, GE Healthcare), equilibrated with 10 mM Tris pH 8.0.

### 1.7 Exonuclease Fluorescence Assay

Recombinant E. coli Exonuclease III was purchased from New England Biolabs (100 units μl⁻¹). Double stranded DNA template labelled with a 5′ fluorophore (5HEX) on the sense strand and a 3′ black hole quencher (BHQ-2a-Q) on the antisense strand was obtained from Operon.

The oligo sequences are given below along with the respective fluorophore and quencher pair:

(SEQ ID NO: 31)
5′[5HEX]GCAACAGAGCTGATGGATCAAATGCATTAGGTAAACATGTT
ACGTCGTAA 3′
(SEQ ID NO: 32)
5′CGATCTTACGACGTAACATGTTTACCTAATGCATTTGATCCATCAGC
TCTGTTGC[BHQ2a]3′

The substrate dsDNA has a 5 bp overhang at the 5′ end of the antisense strand, enabling initiation of exonuclease III on the 3′ end of the sense strand.

Fluorescence measurements were taken using a Cary Eclipse (Varian) with an excitation and emission wavelength of 535 and 554 nm respectively and an excitation and emission slit of 5 nm. Measurements were taken every 4 seconds for 60 minutes. 40 μl reactions were performed at 37° C. and consisted of: 200 nm substrate dsDNA; 25 mM Tris pH 7.5; 1 mM MgCl₂; 100 mM KCl; 0.001 units Exo II; unless otherwise stated.

### 1.8 Planar Bilayer Recordings

All bilayers were formed by apposition of two monolayers of 1,2-diphytanoyl-sn-glycero-3-phosphocholine (Avanti Polar Lipids) across a 60-150 un diameter aperture in Teflon film (25 μm thickness from Goodfellow, Malvern, PA), which divided a chamber into two buffer compartments (cis and trans) each with a volume of 1 ml. Bilayers were formed across the aperture by consecutively raising the buffer level in each compartment until a high resistance seal was observed (≥10 GΩ). Unless otherwise stated, fusion heptamers and DNA or dNMPs were added to the cis compartment, which was connected to ground. The adapter molecule am7βCD or am6-amPDP1-βCD was added to the trans compartment if required, which was connected to the head-stage of the amplifier. Unless stated otherwise, experiments were carried out in 25 mM Tris·HCl, 400 mM KCl pH 8.0, at 22° C.

### 1.9 Exonucleases

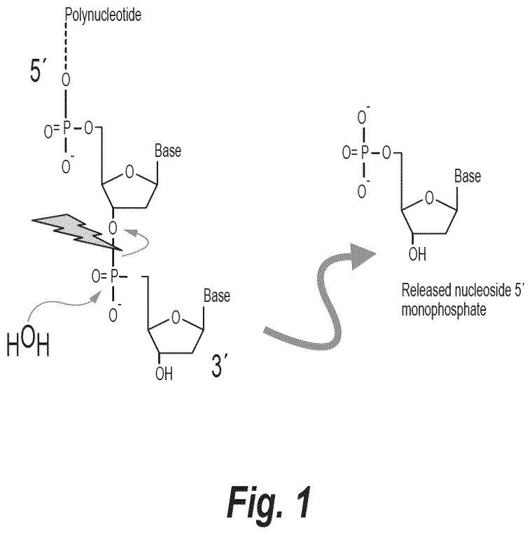

Exonucleases, such as deoxyribonucleases, are a subgroup of the EC 3.1 enzymes. They catalyse the hydrolysis of the phosphodiester bond between adjacent bases in a DNA strand to release individual nucleoside 5′ mono-phosphates (FIG. 1). Attractive activities catalyse the cleavage of this bond (through nucleophilic attack of an activated water molecule upon the phosphorus) as shown.

There are a limited number of distinct enzymatic activities that degrade nucleic acids into their component parts, although numerous homologues will exist in different organisms (for example, Exonuclease III). From a detailed literature search, the two most processive exonuclease enzymes are Exonuclease I, encoded by the sbcB gene of E. coli, and λ-exonuclease, encoded by the exo gene of bacteriophage λ (Thomas, K. and Olivera, B. (1978) Processivity of DNA exonucleases. Journal of Biological Chemistry. 253, 424-429; and Zagursky, R. and Hays, J. (1983). Expression of the phage lambda recombination genes exo and bet under lacPO control on a multi-copy plasmid. Gene. 23, 277-292). In addition, activity of Exonuclease I has been demonstrated in high salt concentrations (Hornblower, B., Coombs, A., Whitaker, R., Kolomeisky, A., Picone, S., Meller, A. Akeson. M. (2007). Single-molecule analysis of DNA-protein complexes using nanopores. Nature Methods. 4, 315-317). As λ exonuclease is a trimer the attachment of a functional exonuclease is more challenging so the monomeric enzyme Exonuclease III was also included, as despite its shorter processivity rate it also degrades one strand of dsDNA to yield nucleoside 5′ monophosphates. Whilst Exo I degrades ssDNA in a 3′-5′ direction RecJ acts 5′-3′ and so was also included in this work (Lovett, S. and Kolodner, R. (1989). Identification and purification of a single-stranded-DNA-specific exonuclease encoded by the recJ gene of Escherichia coli. Proceedings of the National Academy of Sciences of the United States of America. 86, 2627-2631). Both ssDNA exonucleases have been demonstrated to interact and act cooperatively with single stranded binding protein (Genschel, J., Curth, U. and Urbanke, C. (2000) Interaction of E. coli single-stranded DNA binding protein (SSB) with exonuclease I. The carboxy terminus of SSB is the recognition site for the nuclease. Biological Chemistry. 381, 183-192; and Han, E., Cooper, D., Persky, N., Sutera, V., Whitaker, R., Montello, M. and Lovett, S. (2006). RecJ exonuclease: substrates, products and interaction with SSB. Nucleic Acids Research. 34, 1084-1091). The use of these proteins may be required to prevent secondary structure formation of the ssDNA substrate that may enzyme initiation or processivity in high salt concentrations.

Four exonucleases are used in this Example:
1. Exo III from E. coli, Monomeric, dsDNA, 3′-5′ (SEQ ID NOs: 9 and 10)
2. Exo I from E. coli, Monomeric, ssDNA, 3′-5′ (SEQ ID NOs: 11 and 12)
3. RecJ from T. thermophilus, Monomeric, ssDNA, 5′-3′ (SEQ ID NOs: 13 and 14)
4. λ Exo from λ bacteriophage, Trimeric, dsDNA, 5′-3′ (the sequence of one monomer is shown in SEQ ID NOs: 15 and 16)

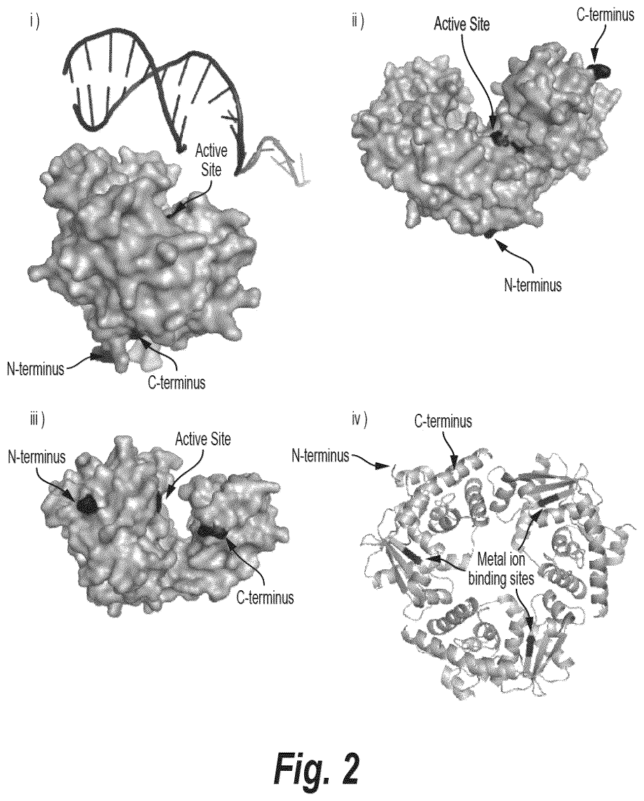

High resolution crystal structures are available for all these enzymes (Mol, C., Kuo, C., Thayer, M., Cunningham, R. and Tainer, J. (1995) Structure and function of the multifunctional DNA-repair enzyme exonuclease III. Nature. 374, 381-386; Kovall, R. and Matthews, B. (1997). Toroidal structure of lambda-exonuclease. Science. 277, 1824-1827; and Busam, R. (2008). Structure of Escherichia coli exonuclease I in complex with thymidine 5′-monophosphate. Acta Crystallographica. 64, 206-210) and are shown in FIG. 2. The TthRecJ is the enzymes core domain as identified by Yamagata et al. (Yamagata, A., Masui, R., Kakuta, Y., Kuramitsu, S. and Fukuyama, K. (2001).

### 1.10 Genetic Attachment

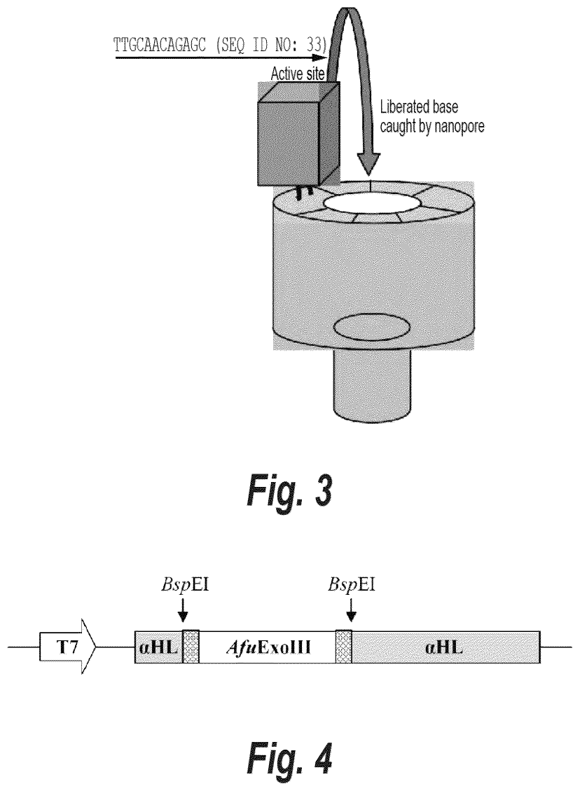

Taking the characteristics of the exonuclease as detailed above, the work described here was guided by the generation of a hypothetical model in which just one of the seven subunits of the αHL heptamer is modified to carry the exonuclease activity. FIG. 3 is a representation of the fusion construct assembled into a heteroheptamer with the exonuclease attached to a loop on the cis side of the protein. This model satisfies other additional desirable characteristics. An exonuclease fused on the cis side of the αHL heptamer under positive potential should release monophosphate nucleosides or ssDNA that will migrate from the cis to the trans side of the pore. This direction of migration is standard in much of the published literature of nanopore sensing. The genetic attachment of an exonuclease within a loop region also invariably means that the N and C terminal linkers can be designed to limit and constrain the mobility of the exonuclease in relation to the lumen of the pore.

In order to create a genetic fusion of the α-HL and the exonuclease proteins, genetic manipulation of the pre-existing expression plasmid pT7-SC1 carrying the wild-type α-HL gene was made (SEQ ID NO: 3). This plasmid carries the gene encoding the wild-type α-HL (SEQ ID NO: 1) without the benefit of any mutations that have been demonstrated to enhance the capacity of the pore to detect and discriminate monophosphate nucleosides. Unique BspEI restriction endonuclease sites were engineered into the α-HL gene at three specific locations, to enable insertion of the exonuclease gene, detailed below. Three plasmids are thus generated, with each one carrying just a single BspEI site for exonuclease gene infusion.

The first insertion site. L1, is located between residues T18 and T19 of the first loop region (N6-V20) of the α-hemolysin protein (SEQ ID NO: 6). The second insertion site, L2, is located between residues D44 and D45 of the start of the second loop region (D44-K50) of the α-hemolysin protein (SEQ ID NO: 7). The third insertion site, L2b, is located between residues K50 and K51 of the end of the second loop region (D44-K50) of the α-hemolysin protein (SEQ ID NO: 8).

Exonuclease genes were codon optimised for expression in E. coli and synthesised by GenScript Corporation (SEQ ID NOs: 10, 12, 15 and 16). Genes were flanked by regions encoding 10 residues of repeating serine-glycine. Such a protein sequence is believed to be substantially devoid of a defined secondary or tertiary structure. The terminal ends of the linkers were also defined by recognition sequences for the restriction endonuclease BspEI, as this sequence also encodes a serine and glycine that form part of the linker. The recognition site of this enzyme (TCCGGA) was similarly engineered into the three specific locations within the αHL gene to provide a means of inserting the exonuclease genes in frame at these defined locations.

The recombinant gene encodes a fusion protein consisting of: a portion of αHL; a 10 serine-glycine linker region: an exonuclease; a 10 serine-glycine linker region; and the remaining portion of αHL. Once made, the chimeric gene construct was sequenced and verified to be as shown in FIG. 4.

Both the N and C-terminii of α-hemolysin are suitable for genetic fusion to an enzyme. It has been shown that the 17 N-terminal residues, which constitute the amino latch, are dispensable for heptamer formation. Whilst it is not possible to delete more than 3 residues from the C-terminus, without effecting oligomerisation, it is already readily presented as a possible attachment point at the back of the cap domain (Walker, B. and Bayley. H. (1995). Key residues for membrane binding, oligomerization and pore-forming activity of Staphylococcal α-hemolysin identified by cysteine scanning mutagenesis and targeted chemical modification. The Journal of Biological Chemistry. 270, 23065-23071).

The attachment of enzymes at the N and C-terminus of α-hemolysin was carried out in a similar manner to that described above. The enzyme and α-hemolysin domains were again mediated by serine-glycine rich linkers to ensure the physical separation necessary for correct folding and spatial separation of each protein domain. The exact details of attachment are however detailed in a later section.

The hemolysin monomers were initially used as a wildtype monomer (wt), however we have shown that a HL-M113R/N139Q monomer shows improved base discrimination and the baseline was changed to this background. Further work showed that the base best resolution was achieved when an adapter molecule was attached to the L135C position, this was added to the hemolysin-exonuclease fusion in later constructs.

In the construct nomenclature, the monomer HL-M113R/N139Q is abbreviated to HL-RQ and the HL-M113R/N139Q/L135C monomer is abbreviated to HL-RQC. Therefore the fusion construct HL-(M113R/N139Q)₆(M I13R/N139Q/L135C-EcoExoIII-L1-H6)₁ is shortened to HL-(RQ)₆(RQC-EcoExoIII-L1-H6)₁.

### 2 Results

### 2.1 Oligomerisation of Loop 1 Fusion Proteins

Water soluble α-hemolysin monomers can bind to and self-assemble on a lipid membrane to form a transmembrane pore of defined structure, via an intermediate heptameric prepore (Walker, B. and Bayley, H. (1995). Key residues for membrane binding, oligomerization and pore-forming activity of Staphylococcal (α-hemolysin identified by cysteine scanning mutagenesis and targeted chemical modification. The Journal of Biological Chemistry. 270, 23065-23071). Fully assembled pores can then be isolated and recovered through SDS PAGE, for biophysical characterisation. Radiolabelled α-hemolysin monomers produced through in vitro transcription translation (IVTT) and oligomerised on purified rabbit red blood cell membranes, enable heptamers to be recovered from the gel using the autoradiograph as template. Modified monomers can also be incorporated into the heptamer in any number and at any of the subunit positions (1-7). The modified subunit also typically carries a poly-aspartate tail to allow the differential migration of homo or heteroheptamers on SDS PAGE for ease of purification for each variant (Braha, O., Walker, B., Cheley, S., Kasianowicz, J., Song, L., Gouaux, J. and Bayley, H. (1997). Designed protein pores as components for biosensors. Chemistry and Biology. 4, 497-505). Due to the size of the exonuclease proteins it was not expected that a poly-aspartate tail would be required on the fusion monomers, as the exonuclease alone should cause a significant shift in electrophoretic mobility to enable identification of individual heteroheptamers away from wild-type homoheptamer.

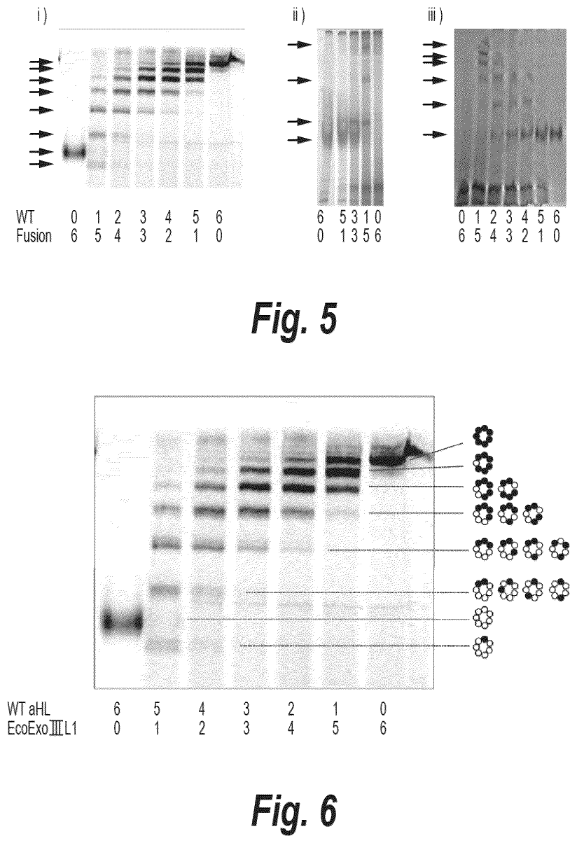

To determine if a mixture of HL-RQ and fusion monomers were able to form heteroheptamers [³⁵S]L-methionine labelled H L-RQ and fusion proteins (HL-wt-EcoExoIII-L1-H6 (SEQ ID NO: 18). HL-RQC-EcoExoIII-L1-H6 (SEQ ID NO: 20), HL-RQC-EcoExoI-L1-H6 (SEQ ID NO: 22) and HL-RQC-TthRecJ-L1-H6 (SEQ ID NO: 24) were expressed by IVTT and oligomerised on purified rabbit red blood cell membranes. The autoradiograph of the gel identified several putative heptamer bands of differing size for all enzyme fusions (FIG. 5).

To characterise these heptamer bands and to identify the ratio of subunits within each, proteins were excised from the gel. Heating heptamer at 95° C. for 10 minutes breaks the protein into its constitutive monomers, which can then be visualised on SDS PAGE for densitometry to determine the heptamer subunit composition. The different characteristic heptamer bands can then be identified as homo or heteroheptamers that consist of different ratios of wild-type and fusion α-HL monomers. This characterisation was performed for putative heptamer bands generated using both the HL-wt-EcoExoIII-L1-H6 and HL-RQC-EcoExoI-L1-H6 fusion proteins.

An importance for a sequencing application is that there preferentially be only one exonuclease moiety, ensuring bases are released only from a single DNA stand being processed at any one time. Electrophoretic migration of a 6:1 HL-monomer:HL-Exonuclease species away from other oligomers is therefore desired for ease of purification. Surprisingly, the HL-(RQ)₆(wt-EcoExoIII-L1-H6)₁ heptamer migrates to a position slightly lower down the gel than HL-(RQ)₇, despite the presence of a ˜36 kDa exonuclease being present on one of the subunits. This band also has a “doublet” appearance, possibly caused by incorrect incorporation of the fusion subunits amino latch due to the downstream insertion of the exonuclease in loop 1 or translation initiating at two points (the start of the fusion protein at hemolysin M1 and also at the first methionine of ExoIII) giving a mixed pool of fusion proteins. The EcoExoIII fusion protein gives formation of all theoretical heteroheptamer varieties and the wild-type and fusion protein homoheptamers. As a significantly smaller protein, ˜36 kDa, and with its N and C terminus co-localised it is perhaps unsurprising that EcoExoI performs better than EcoExoI or TthRecJ as an exonuclease suitable for inserting into loop regions to give good heteroheptamer formation. Both the EcoExoI and TthRecJ fusion proteins give still show formation of heteroheptamers, although with a limited number of fusion monomer subunits, but in contrast the 6:1 heteroheptamer of EcoExoIII these 6:1 heteroheptamers migrate to a position identical to HL-(RQ)₇.

It is an important consideration that by varying the ratio of wild-type to fusion monomer different bands corresponding to the different homo and heteroheptamers were observed. This allows the control of homo or heteroheptamer formation based on the molar ratio of different monomer subunits, which is important for the preferential generation of HL-(RQ)₆(RQ-Exonuclease-H6)₁(FIG. 6).

The conditions for the HL-(RQ)₆(wt-EcoExoIII-L1-H6)₁ heteroheptamer formation were optimised by varying the ratios of monomer proteins. A preferred ratio of 100:1 gives predominately formation of one type of heteroheptamer, HL-(RQ)₆(wt-EcoExoIII-L1-H6)₁, as well as wild-type homoheptamer, HL-(RQ)₇. Affinity purification by the hexa-His tag of the fusion subunit then allows separation of heteroheptamer from HL-RQ homoheptamer.

The HL-(wt-EcoExoIII-L1-H6)₇ homoheptamer and the HL-(RQ)₆(wt-EcoExoIII-L1-H6)₁ heteroheptamer bands were excised from the gel and the protein pores recovered by re-hydration and maceration of the gel slice. These isolated heptamers were both able to insert into planar lipid bilayers to give single channel recordings. The single channel trace for the HL-(wt-EcoExoIII-L1-H6)₇ homoheptamer, however, exhibited numerous blocking events at ≥80 mV. This could be attributed to the presence of seven denatured exonuclease peptide chains surrounding the cap domain, as these events were significantly less pronounced with the HL-(RQ)₆(wt-EcoExoIII-L1-H6)₁ heteroheptamer. The HL-(RQ)₆(wt-EcoExoIII-L1-H6)₁ heteroheptamer gave an open pore current of ˜160 pA and a heteroheptamer containing the mutations necessary for base discrimination HL-(RQ)₆(RQC-EcoExoIII-L1-H6)₁ showed covalent attachment of the β-cyclodexterin adapter molecule, which is characterised by an persistent current block to ˜90 pA.

The construction of a fusion protein involves the linking of two proteins or domains of proteins by a peptide linker. Linker sequence with regard to length, flexibility and hydrophilicity is important so as not to disturb the functions of the domains. The linker regions of loop 1 fusion constructs were initially designed to be of sufficient length to allow the correct folding of both the exonuclease and cx-hemolysin domains of the fusion protein. However, of importance to the release of monophosphate nucleosides in a proximity to the pore lumen is the length and conformation of the linker regions. At some point, however, the linkers will become too short to connect the subunits in their native conformation without strain, which may be particularly detrimental to exonuclease activity and probably oligomerisation. The length of the linkers was therefore reduced to (SG)₄, (SG)₂ and (SG)₁ to determine the effect on oligomerisation efficiency. For oligomerisation the shortened (SG)₄ and (SG)₂ linkers had no adverse effect on the efficiency of heteroheptamer formation. The effect of these shortened linkers on the enzyme activity was not determined but the (SG)₄ fusion protein showed increased expression of soluble protein, which is an indicator of correctly folded proteins.

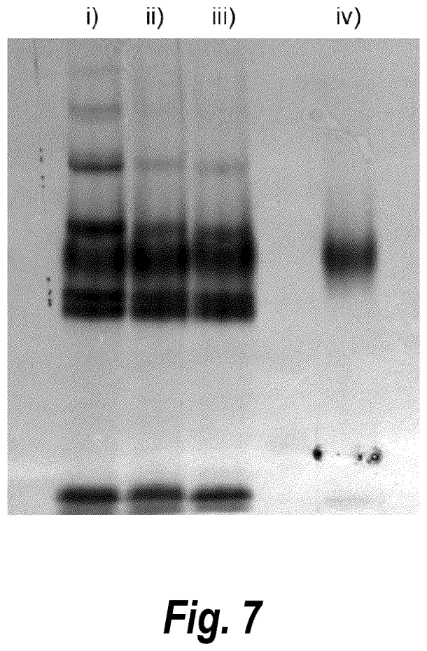

The conformational flexibility of these linkers will also have an effect on the exonuclease position in relation to the pore lumen at any given time. While conformational flexibility may be required at the N and C-terminus linker juncture too much flexibility in the rest of the linker may be detrimental to the co-localisation of the exonuclease active site to the pore lumen. The absence of a β-carbon in glycine permits the polypeptide backbone to access dihedral angles that other amino acids cannot. Proline, as a cyclic imino acid, has no amide hydrogen to donate in hydrogen bonding so cannot fit into either α-helix or β-strand secondary structure. Poly-proline regions are therefore stiff with the absence of secondary structure. By in vivo homologous recombination of PCR products the 10 serine-glycine linker was replaced with 5 proline residues. The use of a rigid polyproline “molecular rulers” was the determined for loop 1 EcoExoIII constructs as the linker between the c-terminus of the exonuclease and the N-terminus of α-hemolysin (FIG. 7).

Heteroheptamer formation was not abolished demonstrating the potential use of polyproline as a linker between the C-terminus of EcoExoIII and α-hemolysin T19 for the fusion protein. Although both fusion proteins showed a lower yield of heteroheptamers where the fusion protein is predominant the formation in particular of HL-(RQ)₆(RQC-EcoExoIII-L1-H6), was unaffected.

The use of different length flexible linkers and alternative rigid linkers for optimising the position and conformational freedom of the exonuclease in relation to the pore lumen, as well as a method for optimising the formation of preferentially 6:1 heteroheptamers, has been demonstrated.

### 2.2 Mutagenesis and Oligomerisation of Loop 2 Fusion Proteins

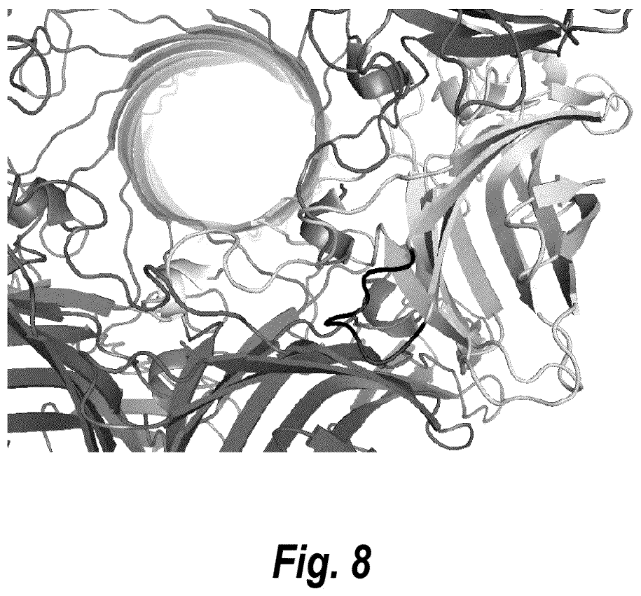

The high yield of heteroheptamers generated by IVTT proteins for the EcoExoIII in loop 1 gave confidence for insertion of EcoExoIII into other loop regions, in particular both positions within loop 2 (FIG. 8). As this loop region connects two integral beta stands then it is likely that any enzymes that do not have a co-localised N and C-terminus will be too disruptive to the α-hemolysin domain, abolishing the ability of this protomer to oligomerise. Only very long linker regions may enable genetic attachment of EcoExoI or TthRecJ at these positions, due to their N and C-terminus localising to domains at distal ends of the respective enzymes.

The oligomerisation of the HL-RQC-EcoExoIII-L2a-H6 and HL-RQC-EcoExoIII-L2b-H6 fusion proteins was poor and only heptamers with an electrophoretic mobility similar to HL-(RQ)₇ and HL-(RQ)₆(RQC-EcoExoIII-L1-H6)₁ were observed. As oligomerisation of HL-RQC-EcoExoIII-L2a-H6 was slightly improved over the HL-RQC-EcoExoIII-L2b-H6 fusion protein, modification was carried out to improve the formation of heteroheptamer. Deletions of residues around the insertion site were made in an attempt to accommodate the terminal linker residues. In addition certain residues in loop 2 may be important for heptamer self-assembly. Sequence alignment of the α-hemolysin monomer with other β-pore forming toxin monomers, LukS and LukF, indicates loop 2 is a highly conserved region and in particular residue D45, which is the residue immediately after the exonuclease linker juncture. The crystal structure of the α-hemolysin heptamer also indicates that H48 is important to binding the amino latch of the adjoining subunit, at position T22 and D24 (Song, L., Hohaugh, M., Shustak, C., Cheley, S., Bayley, H. and Gouaux, E. (1996). Structure of Staphylococcal α-hemolysin, a heptameric transmembrane pore. Science. 274, 1859-1865). Attempts to modify the insertion point to accommodate and characterise these potentially important interactions were therefore made.

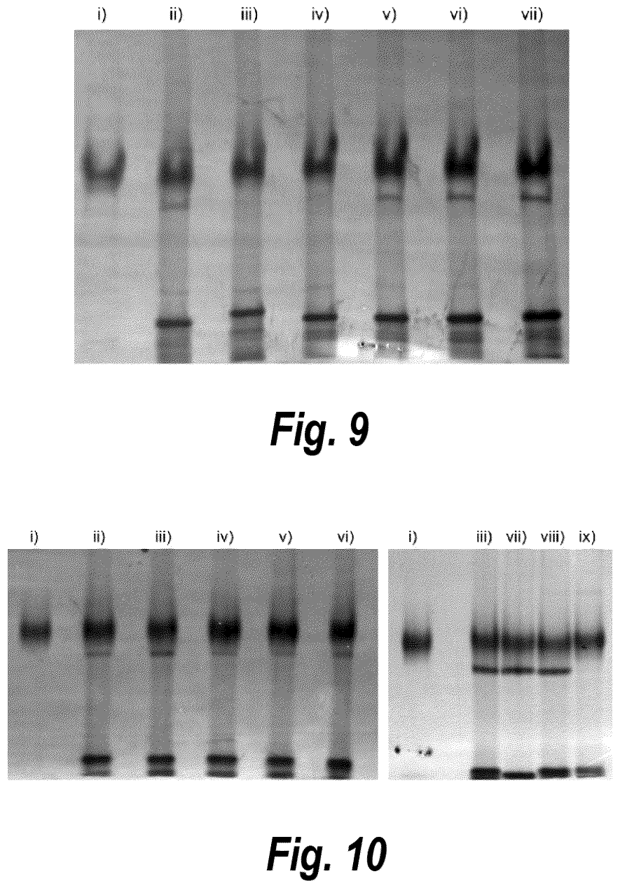

Around the loop 2a EcoExoIII insertion site (D44-D45) residues D45, K46 and N47 were sequentially deleted by in vivo homologous recombination of PCR products. To determine the importance of H48 the site of insertion was also changed to lie between N47-N49, deleting H48 entirely. As previously stated linker flexibility can have an important effect of interaction of domains within a fusion protein. Therefore the flexible 10 serine glycine linkers were replaced with rigid 8 proline linkers in an attempt to confer greater domain separation. Each loop 2 fusion construct was expressed via IVTT and mixed in a 2.5:1 ratio with wild-type in the presence of purified rabbit red blood cell membranes. Any improvement in oligomerisation was determined by densitometry of the autoradiograph (FIG. 9).

Oligomerisation of the L2 fusion protein was abolished when the flexibility of the linker was changed to a more rigid polyproline linker. In addition deletion of H48 and positioning of the exonuclease insertion between N47 and N49 abolished heteroheptamer formation. It appeared that only deletion of residues from around the D44-D45 insertion site improved oligomerisation of the fusion protein. To determine if this could further be improved residue D45 was added back to the loop 2 deletion fusion proteins in a position adjacent to D44, before the EcoExoIII insertion site (FIG. 10).

Heteroheptamer formation was not affected by the position of residue D45 and indeed adding back this residue to all fusion proteins was detrimental to oligomerisation, possibly as it reduced the number of residues deleted to accommodate the exonuclease by one as a consequence. Accommodating the exonuclease is therefore the key to improving the oligomerisation of the loop 2 fusion protein (as in SEQ ID NO: 26). The insertion site was varied further in an attempt to determine how close to the β₂ strand the insertion site could be. The position within the loop region could be important for the relative positioning of the EcoExoIII active site in relation to the pore lumen and it is predicted the closer to β₂ the better the presentation of cleaved monophosphate nucleosides. In each fusion construct the insertion site was not only varied but the following three residues of α-hemolysin at the C-terminus of EcoExoIII were deleted in order to accommodate the exonuclease. Oligomerisation of the alternative loop 2 fusion proteins HL-(RQ)₆(RQC-EcoExoIII-L2-D45-N47Δ-H6)₁, HL-(RQ)₆(RQC-EcoExoIII-L2-F42-D46Δ-H6)₁ and HL-(RQ)₆(RQC-EcoExoIII-L2-I43-D46Δ-H6), determined that the insertion point can lie anywhere within the loop region but as soon as it breaks a region of secondary structure all oligomerisation is abolished (FIG. 10).

Whilst the linkers in the loop 2 fusion protein require some degree of flexibility, as determined by the fact that rigid polyproline linkers could not substitute, the length can be reduced. The linker regions were shortened as for the loop 1 EcoExoIII fusion protein to (SG)₄, (SG)₃, (SG)₂ and (SG)₁ to determine the effect on oligomerisation efficiency. For oligomerisation the shortened (SG)₄, (SG)₃ and (SG)₂ linkers had no adverse effect on the efficiency of heteroheptamer formation. The effect of these shortened linkers on the enzyme activity was not, however, determined.

### 2.3 Genetic Attachment at the N and C-Terminus of α-Hemolysin

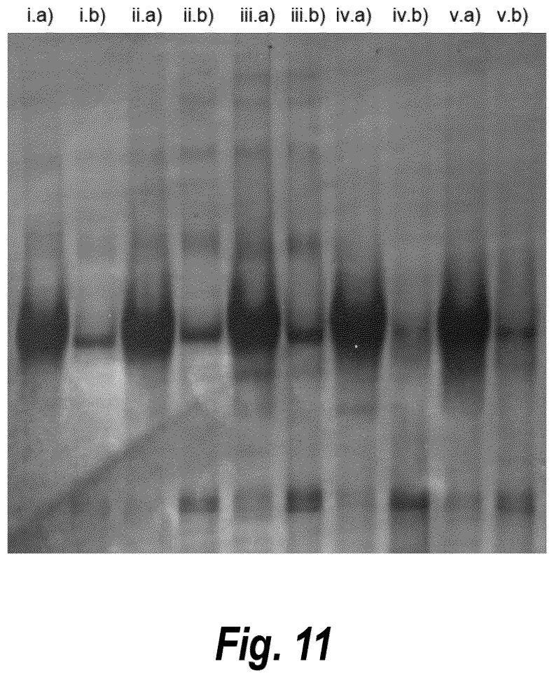

Genetic attachment of two proteins, typically an enzyme to an antibody, has previously focused on the fusion of one protein's C-terminus to another protein's N-terminus, mediated by a peptide linker. As previously mentioned strategies for the attachment of a DNA handling enzyme to the C or N-terminus of α-hemolysin was considered, in particular the attachment of EcoExoI and the Klenow fragment. Attachment of EcoExoI at the C-terminus was mediated by five different linkers in order to determine the optimum fusion protein for oligomerisation. As the C-terminus is at the back of the α-hemolysin cap domain a turn of approximately 180° was desired. In order to initiate this turn either a Gly-Asp or Trp-Pro-Val motif was added at the start of the linker peptide. Two linker peptides were also used, either a flexible 16 serine-glycine or a 12 polyproline. As early results from the EcoExoI loop 1 fusion protein indicated that the 6:1 heteroheptamer had the same electrophoretic mobility as wild-type homoheptamer then a mixture of radiolabelled and non-radio labelled IVTT monomers were used for oligomerisation. Monomers were mixed in a 1:1 ratio and oligomerised on purified rabbit red blood cell membranes (FIG. 11).

Although the predominant fusion protein produced is the 6:1 heteroheptamer this migrates to the same position as the HL-(RQ)₇ homoheptamer. Therefore the proteins corresponding to HL-(RQ)₅(RQC-EcoExoI-Cter-{SG}8-H6)₂. HL-(RQ)₅(RQC-EcoExoI-Cter-DG{SG}8-H6)₂ as well as the HL-(RQ)₅(RQC-EcoExoI-L1-H6)₂ heteroheptamer from an earlier experiment were purified from SDS and the ability to insert into planar lipid bilayers determined. All heteroheptamers were capable of inserting into the lipid bilayer to give single channel recordings.

The success for fusion of the EcoExoI at the C-terminus of α-hemolysin mediated by an (SG)₈ and DG(SG)₈ peptide linker provides the method for the later attachment of other DNA handling enzymes via genetic fusion, such as the Klenow fragment (SEQ ID NOs: 28 and 30). The advantages of the Klenow fragment are the fact it provides a molecular motor for strand sequencing and also shows some resistance to SDS PAGE (Akeson, Personal Communication).

### 2.4 Non-SDS PAGE Purification of Heptamers

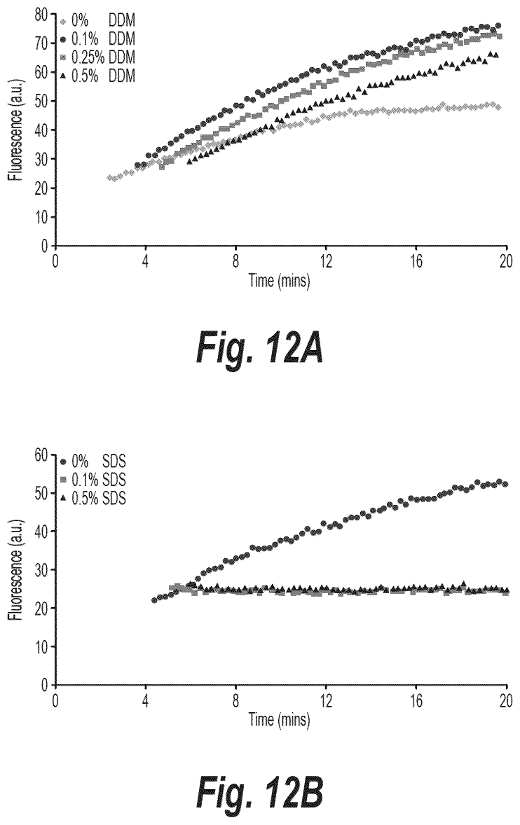

Sodium dodecyl sulphate (SDS) is an anionic surfactant that is highly denaturing to proteins, due to its ability to disrupt non-covalent bonds and bind to the peptide chain. As existing heptamer purification techniques rely on the use of SDS PAGE then the effect of this detergent on EcoExoIII was determined by a fluorescence based activity assay (FIG. 12, left panel).

Even a low concentration of SDS abolished EcoExoIII activity for the native enzyme, making the classical SDS PAGE purification of heptamers denaturing with regard to the exonuclease moiety of a fusion protein heteroheptamer. An alternative purification method was developed therefore using the alternative detergent, n-dodecyl-D-maltopyranoside (DDM). The effect of this surfactant on the EcoExoIII was determined and found to be non-denaturing to the native enzyme (FIG. 12, right panel). Following oligomerisation on rabbit red blood cell membranes instead of purifying heptamers via SDS PAGE the lipid membranes were dissolved by addition of 0.1% DDM for 15 minutes. Heteroheptamers were then purified away from the wild-type homoheptamer by affinity purification to the hexa-His tag on the C-terminus of the fusion protein. A buffer exchange further removed any surfactant and heptamers were then used for single channel recordings. This method does not distinguish entirely between heteroheptamers so the formation of 5:2 was limited by optimising the ratios of monomers mixed.

Purification via DDM extraction produced heptamers that showed an increased number of blocking events and surfactant behaviour on the lipid bilayer in single channel recordings. Whilst the cause of this instability remains undetermined, it is likely to be a result of other membrane proteins released from the rabbit red blood cell membranes, either affecting the lipid bilayer directly or else increasing the protein associated surfactant carryover. Oligomerisation of α-hemolysin monomers is classically facilitated either on purified rabbit red blood cell membranes or deoxycholate micelles. The yield of heptamer from deoxycholate is too poor in this instance to be of use and as previously mentioned the use of purified rabbit red blood cell membranes led to lipid bilayer instability. As an alternative, synthetic lipid vesicles were developed based on the lipid composition of rabbit red blood cell membranes, which lack other the membrane proteins of rabbit red blood cell membranes. These are composed of 30% cholesterol, 30% phosphatidylcholine (PC), 20% phosphatidylethanolamine (PE), 10% sphingomyelin (SM) and 10% phosphatidylserine (PS). The synthetic lipid vesicles developed here give approximately the same efficiency of heptamerisation as observed for rabbit red blood cell membranes. Heptamers purified from these synthetic lipid vesicles by DDM extraction also showed a dramatic decrease in the occurrences of lipid bilayer instability.

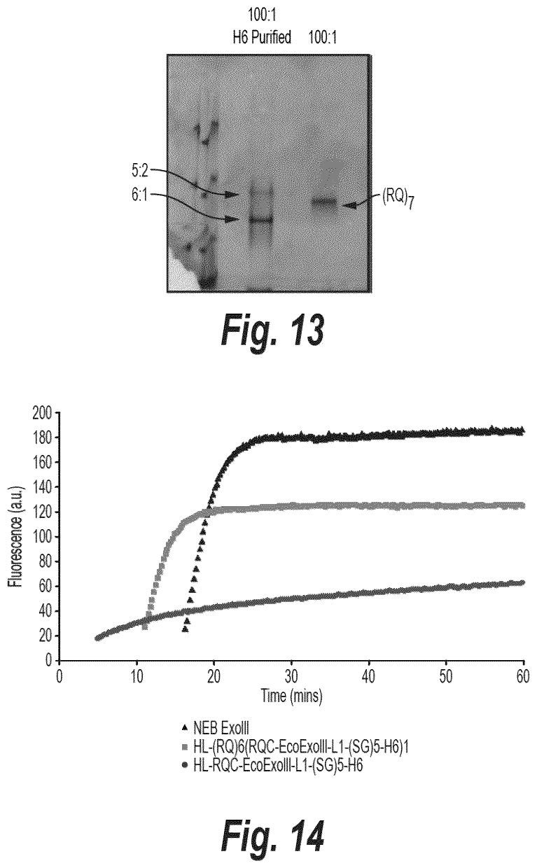

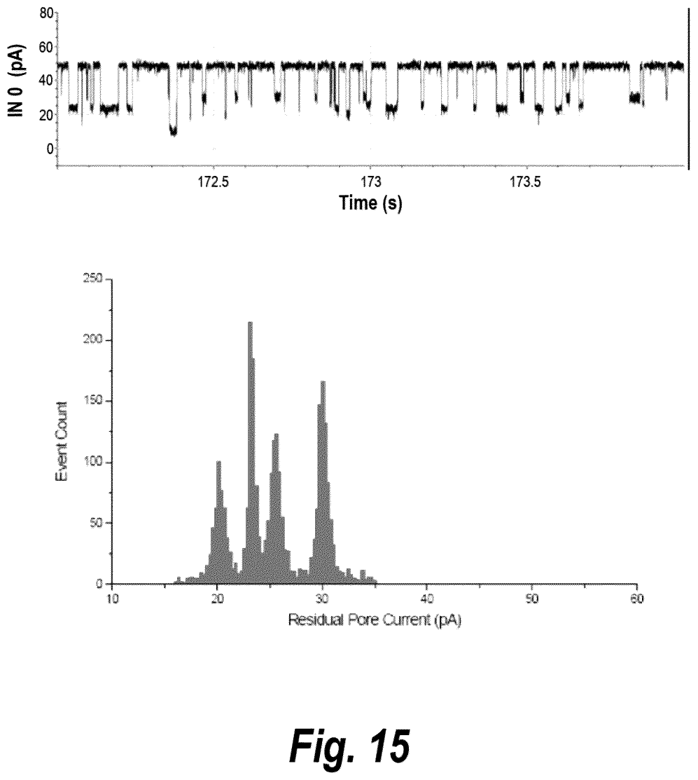

Oligomerisation and DDM purification of heptamers was also determined for E. coli expressed proteins. Expression of wild-type and fusion monomers in E. coli gives a concentration sufficient for large scale production of enzyme pores, typically 3 mg ml⁻¹ and 1 mg ml⁻¹ respectively. Monomers were oligomerised on synthetic lipid vesicles at a ratio of 100:1 (wild-type:fusion) and purified as detailed previously (FIG. 13).

High level E. coli expression of monomers that can be oligomerised on synthetic lipid vesicles was achieved. Purification of the 6:1 heteroheptamer was also achieved in conditions that are non-denaturing to enzymes, ensuring activity of the pores exonuclease moiety.

### 2.5 Enzymatic Activity of Fusion Protein Heptamers

As the terminal ends of the enzyme are conformationally constrained within loop regions of the α-hemolysin monomer then the dynamic movements of the exonuclease domains necessary for activity could be impacted. The native enzyme (Exonuclease HI, NEB)) was able to cleave nucleotides from the dsDNA substrate to a point where the sense strand was no longer of sufficient length to hybridise to the antisense strand (˜8 bp). On dissociation of the DNA strands the fluorophore, at the 5′ end of the sense strand, was sufficiently spatially separated from its quencher pair, at the 3′ end of the antisense strand, giving a fluorescence increase relative to the enzyme activity. The activity of the native enzyme was also determined in a range of salt concentrations (0-1M KCl). Activity of the native enzyme was demonstrated in concentrations ≤300 mM KCl, which is within the experimental conditions required for single channel recordings and base discrimination. To determine if exonuclease activity of the EcoExoIII moiety on the fusion proteins was maintained after genetic attachment and oligomerisation, its activity was determined in this same fluorescence based DNA degradation assay (FIG. 14).

The EcoExoIII fusion proteins demonstrated retained exonuclease activity but as yet this is a qualitative rather than quantitative indication as amount of fusion protein was not determined. Therefore the effect of genetic fusion of the EcoExoIII to an α-hemolysin monomer on the rate of exonuclease activity cannot be determined as yet.

The exonuclease activity of the fusion protein was checked at all stages of purification and found to retain activity. Following oligomerisation and DDM purification the activity of fully formed pores was also checked and found to show some exonuclease activity. This demonstrates the ability to genetically couple an enzyme to a protein pore and still retain activity of the enzyme after expression and oligomerisation to a fully assembled pore.

### 2.6 Pore Forming Activity of Fusion Protein Heptamers

As previously mentioned in the text the ability of a variety of different enzyme pore constructs to insert into lipid bilayers for single channel recordings has been shown. We have demonstrated that changes to the β-barrel of the α-hemolysin protein can enable covalent linkage and stabilisation of an adapter molecule for continuous base detection. For this the pore preferentially requires 6 subunits with mutations M113R/N139Q and 1 subunit with mutations M113R/N139Q/L135C. To determine if the exonuclease domain of the fusion protein within loop regions affected the ability of the pore to discriminate bases the M113R/N139Q/L135C mutations were made in the fusion constructs. As base discrimination preferentially requires a heteroheptamer with only one subunit carrying the L135C mutation and the enzyme pore preferentially one subunit being a fusion protein, the L135C mutation was made in the fusion protein. The wild-type M113R and N139Q construct from previous work was used for the other subunits. E. coli expressed HL-RQ and HL-RQC-EcoExoIII-L2-D46-N47Δ-H6 were oligomerised on synthetic lipid vesicles (at a ratio of 100:1) and purified by DDM extraction. The exonuclease activity of the fully formed pore was determined and indicated correct folding of the exonuclease moiety. The protein was also used for electrophysiology to determine firstly pore functionality and secondly if base discrimination was possible (FIG. 19).

The 6:1 heteroheptamer can be inserted into a lipid bilayer and a stable transmembrane current established. This current can be modulated by the introduction of β-cyclodexterin, and is further reduced by the addition of monophosphate nucleosides. The presence of the exonuclease domain appears to have no detrimental effect on current flow or the base discrimination by the pore. Although the work shown is for a heteroheptamer incorporating a fusion protein with the insertion of EcoExoIII at the loop 2 position, similar data was acquired for the loop 1 heteroheptamers.

1.-41. (canceled)

42. A method of sequencing a target nucleic acid sequence, comprising:
(a) contacting the target nucleic acid sequence with a transmembrane pore comprising a transmembrane protein pore subunit that is covalently attached to a nucleic acid handling enzyme such that the nucleic acid handling enzyme pushes or pulls the target nucleic acid sequence through the transmembrane pore and a proportion of the nucleotides in the target nucleic acid sequence interacts with the transmembrane pore; and
(b) measuring the current passing through the transmembrane pore during each interaction and determining the sequence of the target nucleic acid sequence.

43. The method of claim 42, wherein the target nucleic acid sequence comprises a double-stranded DNA sequence.

44. The method of claim 42, wherein the target nucleic acid sequence comprises a single-stranded RNA sequence.

45. The method of claim 42, wherein a potential is applied across the transmembrane pore.

46. The method of claim 45, wherein the nucleic acid handling enzyme pushes or pulls the target nucleic acid sequence from a cis side of the transmembrane pore to a trans side of the transmembrane pore.

47. The method of claim 45, wherein the nucleic acid handling enzyme pushes or pulls the target nucleic acid sequence from a trans side of the transmembrane pore to a cis side of the transmembrane pore.

48. The method of claim 42, wherein the nucleic acid handling enzyme is a polymerase, exonuclease, helicase or topoisomerase.

49. The method of claim 42, wherein the nucleic acid handling enzyme is a DNA polymerase or RNA polymerase.

50. The method of claim 42, wherein the transmembrane protein pore subunit is from a β-barrel pore or an α-helix pore.

51. The method of claim 42, wherein the transmembrane protein pore subunit is from α-hemolysin.

52. The method of claim 42, wherein the transmembrane protein pore subunit comprises an amino acid sequence having at least 70% homology to SEQ ID NO: 2.

53. The method of claim 42, wherein the transmembrane protein pore subunit comprises an amino acid sequence having at least 80% homology to SEQ ID NO: 2.

54. The method of claim 42, wherein the transmembrane protein pore subunit comprises an amino acid sequence having at least 90% homology to SEQ ID NO: 2.

55. The method of claim 42, wherein the transmembrane protein pore subunit comprises an amino acid sequence having at least 95% homology to SEQ ID NO: 2.

56. The method of claim 54, wherein the nucleic acid handling enzyme is attached to the transmembrane protein pore subunit at a position corresponding to an amino acid position of SEQ ID NO: 2 selected from the group consisting of positions 1, 7 to 21, 31 to 34, 45 to 51, 63 to 66, 72, 92 to 97, 104 to 111, 124 to 136, 149 to 153, 160 to 164, 173 to 206, 210 to 213, 217, 218, 223 to 228, 236 to 242, 262 to 265, 272 to 274, 287 to 290, and 294.

57. The method of claim 42, wherein the transmembrane pore comprises 6, 7 or 8 transmembrane protein pore subunits.

58. The method of claim 42, wherein the nucleic acid handling enzyme is attached to the transmembrane protein pore subunit through a linker.

59. The method of claim 58, wherein the linker comprises a peptide linker or a chemical linker.

60. The method of claim 42, wherein the at least one transmembrane protein pore subunit is covalently attached to the carboxyl terminus of the nucleic acid handling enzyme.
